SEQUENCE LISTING (2 sequences)

SEQ ID NO: 1 - protein, 5 aa, Artificial sequence
  synthetic sequence
VAKGL

SEQ ID NO: 2 - protein, 441 aa, Artificial sequence
  synthetic sequence
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

(19) United States
(12) Patent Application Publication
(10) Pub. No.: US 2014/0094482 A1
(43) Pub. Date: Apr. 3, 2014

(54) BENZAZOCINE-RING COMPOUND INHIBITION OF TAU HYPERPHOSPHORYLATION

(71) Applicants: George B. Thornton, Austin, TX (US); Hoau-Yan Wang, Philadelphia, PA (US); Lindsay Burns Barbier, Austin, TX (US); Michael S. Zamloot, Austin, TX (US)

(72) Inventors: George B. Thornton, Austin, TX (US); Hoau-Yan Wang, Philadelphia, PA (US); Lindsay Burns Barbier, Austin, TX (US); Michael S. Zamloot, Austin, TX (US)

(21) Appl. No.: 13/968,078

(22) Filed: Aug. 15, 2013

Related U.S. Application Data
(60) Provisional application No. 61/684,429, filed on Aug. 17, 2012.
(60) Provisional application No. 61/684,041, filed on Aug. 16, 2012.

(51) Int. Cl. A61K 31/485; C12N 5/079

(52) U.S. Cl. CPC A61K 31/485; C12N 5/0618
USPC 514/282; 435/375; 514/289

(57) ABSTRACT

A method of inhibiting hyperphosphorylation of the tau protein and/or a TLR4-mediated immune response is disclosed. The method contemplates administering to cells in recognized need thereof such as cells of the central nervous system a FLNA-binding effective amount of a FLNA-binding benzazocine-ring compound or a pharmaceutically acceptable salt thereof such as a) an opioid receptor antagonist compound or b) a mixed opioid receptor agonist and antagonist (agonist/antagonist) compound, c) an opioid receptor agonist compound or d) an enantiomer of an opioid receptor interacting compound, that binds to a pentapeptide of filamin A (FLNA) of SEQ ID NO: 1. The administered compound preferably contains at least four of the six pharmacophores of FIGS. 5-10.

### CROSS-REFERENCE TO RELATED APPLICATIONS

This application claims priority to U.S. application Ser. No. 61/684,429, filed on Aug. 17, 2012, and to application Ser. No. 61/684,041, filed on Aug. 16, 2012, both of whose disclosures are incorporated herein by reference.

### TECHNICAL FIELD

The present invention contemplates a method of central nervous system (CNS) treatment to inhibit the formation of hyperphosphorylated tau protein and the use of a contemplated compound in the manufacture of a medicament for inhibiting tau protein hyperphosphorylation that can lead to pathological formation of neurofibrillary tangles (NFTs). The method and use also lead to the enhancement of function of one or more of the alpha-7 nicotinic acetylcholine receptor (α7nAchR), the insulin receptor and the N-methyl-D-aspartate receptor.

### BACKGROUND ART

The microtubule-associated protein tau (MAPT) occurs mostly in axons and in lesser amounts in astrocytes and oligodendrocytes, and stabilizes neuronal microtubules for their role in the development of cell processes, establishing cell polarity and intracellular transport. A single gene encodes a tau protein with an open reading frame that can encode 758 amino acid residues. Tau is listed in the UniProtKB/Swiss-Prot data base under the designation P10636. At least nine alternative splicing isoforms are recognized in the UniProtKB/Swiss-Prot data base.

Early work by Goedert and co-workers identified six isoforms that contain 352 to 441 amino acid residues [Mandelkow et al., Trends in Cell Biology, 8:425-427 (1998); see also, Johnson et al., J. Cell Sci, 117(24):5721-5729 (2004)]. The numbering of the amino acid residue sequence and the phosphorylation positions referred to herein is done in line with the human tau isoform referred to as “htau 40” in Goedert et al., Neuron 3:519-526 (1989). That 441 residue tau isoform is also referred to as Tau-4 or Tau-F in the UniProtKB/Swiss-Prot data base in which it is given the designation P10636-8. The amino acid residue sequence of TAU-4 (P10636-8, htau 40) is shown in SEQ ID NO: 2.

Tau is a substrate for a number of kinase enzymes [Johnson et al., J Cell Sci, 117(24):5721-5729 (2004)]. Phosphorylation at serine and threonine residues in S-P or T-P motifs by proline-directed protein kinases (PDPK1: CDK1, CDK5, GSK3, MAPK) and at serine residues in K-X-G-S motifs by MAP/microtubule affinity-regulating kinase (MARK1 or MARK2) are frequently found.

An enzyme of that group, glycogen synthase kinase 3β (GSK3β), can be a predominant tau kinase [Cho et al., J. Neurochem, 88:349-358 (2004)]. GSK3β can phosphorylate unprimed sites that are in proline-rich regions (Thr-181, Ser-184, Ser-262, Ser-356 and Ser-400) or unprimed sites (Ser-195, Ser-198, Ser-199, Ser-202, Thr-205, Thr-231, Ser-235, Ser-262, Ser-356 and Ser-404) where a serine or threonine is prephosphorylated by another protein kinase (e.g., A-kinase) at a site that is located four amino acid residues C-terminal to the GSK3β site [Cho et al., J. Neurochem, 88:349-358 (2004); Wang et al., FEBS Lett, 436:28-34 (1998)].

The normophosphorylated form of the protein is a microtubule-associated protein that stimulates and stabilizes microtubule assembly. That normophosphorylated form typically contains two-three moles of phosphate per mole of protein [Kickstein et al., Proc Natl Acad Sci, USA, 107(50):21830-21835 (2010)].

Multiply phosphorylated (hyperphosphorylated) tau proteins; i.e., tau proteins that contain more than the normophosphorylated number of phosphate groups, can result in the formation of neurofibrillary tangles that are associated with several pathological conditions that are referred to collectively as tauopathies. For example, tau phosphorylation levels in Alzheimer's disease patients are three- to four-fold higher than the number of phosphate groups present in the normophosphorylated molecule [Kickstein et al., Proc Natl Acad Sci, USA, 107 (50):21830-21835 (2010)].

Increasing evidence suggests that neuroinflammation is a common feature of tauopathies. Thus, activated microglia are found in the postmortem brain tissues of various human tauopathies including Alzheimer's disease (AD), frontotemporal dementia (FTD), progressive supranuclear palsy and corticobasal degeneration [Gebicke-Haerter, Microsc Res Tech, 54:47-58 (2001); Gerhard et al., Mov Disord, 21:89-93 (12006); Ishizawa et al., J Neuropathol Exp Neurol, 60:647-657 (2001)].

Induction of systemic inflammation via administration of the Toll-like receptor 4 (TLR4) ligand, lipopolysaccharide (LPS), significantly induces MAPT hyperphosphorylation in a triple transgenic mouse model of AD [Kitazawa et al., J Neurosci, 25:8843-8853 (2005)]. The immunosuppressant drug FK506 (tacrolimus) attenuated microglial activation and extended the life span of P301S transgenic mouse model of FTD [Yoshiyama et al., Neuron 53:337-351 (2007)]. Further, a growing number of studies suggest that proinflammatory cytokines, such as interleukin-1 (IL-1), interleukin-6 (IL-6), and nitric oxide released from astrocytes can accelerate MAPT pathology and formation of neurofibrillary tangles (NFTs) in vitro [Li et al., J Neurosci, 23:1605-1611 (2003); Quintanilla et al., Exp Cell Res, 295:245-257 (2004); Saez et al., In Vivo, 18:275-280 (2004)].

The toll-like receptors (TLRs) are a group of transmembrane receptors whose cytoplasmic portions are highly similar, having a high similarity to the interleukin-1 (IL-1) receptor. That cytoplasmic portion is now referred to as the Toll/IL-1 receptor (TIR) domain. The extracellular portions are structurally unrelated. The TLRs recognize pathogen components. [Takeda et al., Seminars in Immunology, 16:3-9 (2004).]

TLR4 plays a fundamental role in pathogen recognition in recognizing lipopolysaccharide (LPS) found in most gram-negative bacteria as well as other molecules. This receptor also plays a role in activation of innate immunity. TLR4 pathway activation can be an indicator of an infection.

TLR4 typically associates with the adapter molecule, MD2, CD14 and the lipopolysaccharide binding molecule (LPB) when associating with LPS. Signaling occurs through a series of cytoplasmic molecules in what are referred to as the myeloid differentiation factor 88-(MyD88-) dependent pathway common to all TLRs, and the MyD88-independent pathway shared by TLR3 and TLR4. TLR3 recognizes double-stranded RNA and its activation occurs under different conditions from TLR4 activation.

Signaling induced by LPS via the MyD88-independent pathway leads to activation of the transcription factor IRF-3, and thereby induces IFN-β. IFN-β, in turn, activates Stat1, leading to the induction of several IFN-inducible genes. LPS-induced activation of NF-κB and JNK appears to be independent of the presence of MyD88. [Takeda et al., Seminars in Immunology, 16:3-9 (2004).]

TLR4 is present in cells of the immune system such as B cells, T cells and macrophages, as well as cells of the CNS. TLR4 is an important mediator of the innate immune response, and significantly contributes to neuroinflammation induced by brain injury. The TLR4-mediated neuroinflammation typically proceeds through the TLR4/adapter protein MyD88 signaling pathway.

Mao et al., J Neurotrauma, May 14 (2012) reported the potential neuroprotective mechanisms of pituitary adenylate cyclase-activating polypeptide-(PACAP-) pretreatment in a rat model of traumatic brain injury (TBI). It was found that TBI induced significant upregulation of TLR4 with peak expression occurring 24 hours post-trauma.

Pretreatment with PACAP significantly improved motor and cognitive dysfunction, attenuated neuronal apoptosis, and decreased brain edema. That pretreatment inhibited TLR4 upregulation as well as that of its downstream signaling molecules, MyD88, p-IκB, and NF-κB, and suppressed increases in levels of the downstream inflammatory agents, interleukin-1β (IL-1β) and tumor necrosis factor-α (TNF-α), in the brain tissue around the injured cortex and in the hippocampus. PACAP treatment thus exerted a neuroprotective effect in this rat model of TBI, potentially via inhibiting a secondary inflammatory response mediated by the TLR4/MyD88/NF-κB signaling pathway in microglia and neurons, thereby reducing neuronal death and improving the outcome following TBI.

Traumatic brain injury (TBI) is also the “signature” injury of recent military conflicts and is associated with psychiatric symptoms and long-term cognitive disability. Chronic traumatic encephalopathy (CTE), a hyperphosphorylated tau protein-linked neurodegenerative disorder (tauopathy) reported in athletes with multiple concussions, shares clinical features with TBI in military personnel exposed to explosive blast. CTE also shares pathology found in boxers that was previously known as dementia pugilistica. [Gandy et al., Sci. Transl. Med. 4:1341-1343 (May 12, 2012).]

Goldstein et al., Sci. Transl. Med. 4:134ra60 (2012) investigated this connection between TBI and CTE in a series of postmortem brains from U.S. military veterans with blast exposure and/or concussive injury. Those authors reported evidence for CTE neuropathology in the military veteran brains that is similar to that observed in the brains of young amateur American football players and a professional wrestler. The investigators developed a mouse model of blast neurotrauma that mimics typical blast conditions associated with military blast injury and discovered that blast-exposed mice also demonstrate CTE neuropathology, including tau protein hyperphosphorylation, myelinated axonopathy, microvascular damage, chronic neuroinflammation, and neurodegeneration.

The mouse neuropathology was reported to be accompanied by functional deficits, including slowed axonal conduction, reduced activity-dependent long-term synaptic plasticity, and impaired spatial learning and memory that persisted for 1 month after exposure to a single blast. The investigators then showed that blast-induced learning and memory deficits in the mice were reduced by immobilizing the head during blast exposure.

Neuropathological findings in the military veterans with blast exposure and/or concussive injury and young-adult athletes with repetitive concussive injury were consistent with those authors' previous CTE case studies [McKee et al., J Neuropathol Exp Neurol 68:709-735 (2009); McKee et al., J Neuropathol Exp Neurol 69:918-929 (2010)], and were reported to be readily differentiated from neuropathology associated with Alzheimer's disease, frontotemporal dementia, and other age-related neurodegenerative disorders.

Apolipoprotein E (ApoE) is a class of apolipoprotein found in the chylomicron and intermediate-density lipoprotein (IDLs) that binds to a specific receptor on liver cells and peripheral cells. ApoE has been studied for its role in several biological processes not directly related to lipoprotein transport, its more-studied function, including Alzheimer's disease, immunoregulation, and cognition.

ApoE is 299 amino acids long and transports lipoproteins, fat-soluble vitamins, and cholesterol into the lymph system and then into the blood. It is synthesized principally in the liver, but has also been found in other tissues such as the brain. In the nervous system, non-neuronal cell types, most notably astroglia and microglia, are the primary producers of ApoE, whereas neurons preferentially express the receptors for ApoE.

There are seven currently identified mammalian receptors for ApoE that belong to the evolutionarily conserved low density lipoprotein receptor gene family. ApoE is a polymorphic gene with three major isoforms, ApoE2, ApoE3, ApoE4, which translate from three alleles of the gene, of which ApoE-ε3 is the “normal” allele, and ApoE-ε2 and ApoE-ε4 are dysfunctional alleles.

ApoE4 has been implicated in atherosclerosis and Alzheimer's disease, impaired cognitive function, and reduced neurite outgrowth. The ApoE4 variant is the largest known genetic risk factor for late-onset sporadic Alzheimer's Disease (AD) in a variety of ethnic groups. Caucasian and Japanese carriers of two E4 alleles have between 10 and 30 times the risk of developing AD by 75 years of age, as compared to those not carrying any E4 alleles.

Although 40-65% of AD patients have at least one copy of the 4 allele, ApoE4 is not a determinant of the disease—at least one-third of patients with AD are ApoE4 negative and some ApoE4 homozygotes never develop the disease. However, those with two E4 alleles have up to 20 times the risk of developing AD.

In addition, Apo E4 overexpression in mouse neurons resulted in hyperphosphorylation of tau and the development of motor problems, accompanied by muscle wasting, loss of body weight and premature death. [Tesseur et al., Am J Pathol, 156(3):951-964 (2000).] On the other hand, treatment of neurons with exogenously supplied Apo E isoforms (E2 or E4) affects several downstream signaling cascades in neurons: decreased tau kinase phosphorylation and inhibition of tau phosphorylation at Thr171 and Ser202/Thr205 epitopes in the primary neuronal culture. ApoE can alter levels of tau kinases and phospho-tau epitopes, potentially affecting tau neuropathological changes seen in AD brains. [Hoe et al., Molecular Degeneration, 1:8 (2006).]

Eisenberg and co-workers have studied the formation of beta-sheet fibrils from self-aggregating tau protein, and found that a particular hexapeptide can inhibit their formation by interfering with the ‘steric zipper’ of the beta-sheet fibril. However, the inhibiting peptide is too large to penetrate deeply into the brain nor does it appear to penetrate the brain cells in which the tau fibrils form. See, Sawaya et al., Nature, 447:453-457 (2007); Landau et al., PLoS Biology, 9(6):e1001080 (2011); and Sievers et al., Nature, DOI:10.1038/nature10154 (2011). It would therefore be beneficial if an inhibitor of the formation of tau-containing NFTs could be found that penetrates the brain and other CNS structures, as well as the cells of those structures.

Alzheimer's disease (AD) poses a huge unmet medical need, with an estimated 35 million current patients worldwide and no disease-modifying treatment available. The two classes of drugs currently used for AD, cholinesterase inhibitors and memantine only transiently enhance cognitive function in these patients.

The causative agent in AD pathology is generally accepted to be amyloid-β (Aβ), or particularly Aβ₄₂. Aβ is a 39-42-residue proteolysis product of amyloid precursor protein (APP) that is an integral membrane protein expressed in many tissues and concentrated in the synapses of neurons.

Transgenic animals with increased levels of Aβ can model AD, and Aβ levels in postmortem AD brains are correlated with the degree of cognitive impairment and neuropathology. [Tanzi et al., Cell 120:545-555 (2005).] This correlation is higher for soluble Aβ than for Aβ-rich plaques, implicating soluble Aβ in AD pathogenesis. [Naslund et al., J Am Med Assoc, 283:1571-1577 (2000).]

It is believed that the critical pathogenic role of soluble Aβ is toxic signaling via the α-7 nicotinic acetylcholine receptor (α7nAChR), as demonstrated a decade ago. Aβ binds this receptor with high affinity [Wang et al., J Biol Chem 275:5626-5632 (2000); Wang et al., J Neurochem 75:1155-1161 (2000)], activating ERK2, which phosphorylates the tau protein [Wang et al., J Biol Chem 278:1547-31553 (2003)]. ERK2 is also known as mitogen-activated protein kinase 1 (MAPK1).

Persistent abnormal hyperphosphorylation of tau proteins results in neurofibrillary tangles (NFTs), a prominent neuropathological feature in AD brain, and the magnitude of these lesions correlates with the severity of AD symptoms [Delacourte et al., Neurology 52:158-1165 (1999); Delacourte et al., Neurology 43:93-204 (1998).] The NFTs are initially intracellular, and become extracellular ghost tangles after death of the neuron [Mandelkow et al., Trends in Cell Biology, 8:425-427 (1998)].

Aβ peptide has been shown to induce tau phosphorylation in several in vitro experimental systems [Johnson et al., J Alzheimers Dis 1:29-351 (1999)], and Aβ-induced tau phosphorylation has been demonstrated to be dependent on α7nAChR, because pretreatment of tissues with α7nAChR antagonists or with Aβ₁₂₋₂₈, which inhibit the Aβ₄₂-α7nAChR interaction, reduces Aβ₄₂-induced tau phosphorylation [Wang et al., J Biol Chem 278:547-31553 (2003)].

Phosphorylation of some sites appear to regulate microtubule-binding properties (e.g., Ser262 and Ser356) [Mandelkow et al., Trends in Cell Biology, 8:425-427 (1998)]. On the other hand, phosphorylation at one or more of 202Ser, 231Thr and 181Thr is found in tau-containing NFTs [Wang et al., J Biol Chem 278:31547-31553 (2003); Wang et al., Biol Psychiatry 67:522-530 (2010)].

The critical role of the α7nAChR in mediating neurofibrillary pathology is further supported by at least two findings: 1) protracted incubation of Aβ₄₂ with SK-N-MC cells that over-express α7nAChRs promotes NFTs, and 2) antisense-α7nAChR oligonucleotides that reduce α7nAChR levels abolish Aβ₄₂-induced neurofibrillary lesions [Wang et al., J Biol Chem 278:31547-31553 (2003)]. These data suggest that chronic perturbation of the α7nAChRs with Aβ₄₂ in AD brains leads to neurofibrillary phosphorylated tau-containing lesions.

As discussed in detail hereinafter, the present invention provides a method to inhibit Aβ₄₂-induced hyperphosphorylation of tau proteins by inhibiting one or more signaling pathways that utilize the signaling scaffold, filamin A (FLNA). In one pathway, Aβ and α7nAChR interact leading to the recruitment of FLNA. In another pathway, TLR4 activated by Aβ₄₂ or its cognate ligand, LPS for example, and the TLR4-mediated signaling is activated through the recruitment of FLNA to the TLR4 receptor. That Aβ₄₂ induces FLNA recruitment to α7nAChR or TLR4 as well as tau phosphorylation can be observed by incubating 250,000 cells in 250 μl of oxygenated Kreb's-Ringer with 1 nM Aβ₄₂. This Aβ₄₂-mediated effect was found to be plateaued at 100 nM.

The treatment approach disclosed below is targeted at inhibiting hyperphosphorylation of tau proteins mediated by FLNA using a compound that binds FLNA with high affinity. This high affinity compound-FLNA binding is believed to alter the conformation of FLNA and prevent it from interacting with other signaling molecules such as α7nAChRs, thereby inhibiting the hyperphosphorylation of the tau protein.

### BRIEF SUMMARY OF THE INVENTION

The present invention contemplates a method of inhibiting hyperphosphorylation [phosphorylation at one or more of serine-202 (also 202Ser and S²⁰²), threonine-231 (also 231Thr and T²³¹) and threonine-181 (also 181Thr and T¹⁸¹) in addition to phosphorylation that may be present at any other site] of the tau protein. A contemplated method comprises the steps of administering to central nervous system cells in recognized (diagnosed) need thereof a FLNA-binding effective amount of a FLNA-binding 2,6-methano-3-benzazocine-ring compound or a pharmaceutically acceptable salt thereof that binds to filamin A or binds to a pentapeptide of filamin A (FLNA) of SEQ ID NO: 1 as described in Example 1 that is hereinafter generally referred to as a “benzazocine-ring compound”. A 2,6-methano-3-benzazocine ring structure present in a benzazocine-ring compound is shown below without substituents. A benzazocine-ring

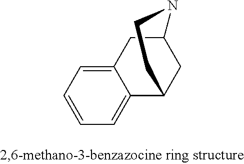

compound typically interacts with an opioid receptor and can be a) an opioid receptor antagonist, b) mixed agonist/antagonist, c) an opioid receptor agonist compound or d) an enantiomer of an opioid receptor interacting compound. In binding to a FLNA pentapeptide of SEQ ID NO: 1, a benzazocine-ring compound inhibits at least about 60 percent and more preferably at least about 70 percent of the FITC-labeled naloxone binding to that FLNA pentapeptide when present at a 10 μM concentration and using unlabeled naloxone as the control inhibitor at the same concentration.

A contemplated FLNA-binding benzazocine-ring compound preferably contains at least four of the six pharmacophores of FIGS. 5-10. One preferred FLNA-binding benzazocine-ring compound is a morphinan or 4,5-epoxymorphinan ring compound, as are well known, whereas another is a benzomorphan compound as is also well known. An opioid receptor with which a contemplated benzazocine-ring compound interacts is preferably the mu opioid receptor (MOR).

The use of a single stereoisomer or mixture of stereoisomers, or a pharmaceutically acceptable salt of a contemplated stereoisomeric compound is also contemplated. The contemplated administration can take place in vivo or in vitro, and is typically repeated when administered in vivo. For that in vivo use, administration of a FLNA-binding benzazocine-ring compound is typically repeated a plurality of times and can be discontinued when the amount of tau protein phosphorylated at one or more of the three protein sequence positions (S²⁰², T²³¹, and T¹⁸¹) becomes constant.

Another aspect of the invention contemplates a method of inhibiting a TLR4-mediated immune response such as inflammation of cells of the CNS. A contemplated method comprises administering to TLR4-containing cells in recognized (diagnosed) need thereof a FLNA-binding effective amount of a FLNA-binding benzazocine-ring compound or a pharmaceutically acceptable salt thereof such as a) an opioid receptor antagonist, b) a mixed agonist/antagonist, c) an opioid receptor agonist compound or d) an enantiomer of an opioid receptor interacting compound, such as a morphinan or 4,5-epoxymorphinan ring compound or benzomorphan ring compound, that binds to a pentapeptide of filamin A (FLNA) of SEQ ID NO: 1. A contemplated FLNA-binding benzazocine-ring compound exhibits at least about 60 percent and more preferably about 70 percent of the FITC-labeled naloxone binding when present at a 10 μM concentration and using unlabeled naloxone as the control inhibitor at the same concentration. A contemplated FLNA-binding benzazocine-ring compound preferably contains at least four of the six pharmacophores of FIGS. 5-10. It is again presently preferred to utilize a compound that exhibits the lesser opioid receptor activity (antagonist or agonist) over a more active enantiomer for the present method.

The use of a single stereoisomer or mixture of stereoisomers, or a pharmaceutically acceptable salt of a contemplated stereoisomeric compound is also contemplated. The contemplated administration can take place in vivo or in vitro, and is typically repeated when administered in vivo to the cells of a host animal such as a human.

In one aspect of an above method, tau hyperphosphorylation of one or more of S²⁰², T²³¹ and T¹⁸¹ occurs through the interaction of Aβ and α7nAChR via the scaffolding protein filamin A (FLNA). In another aspect of a contemplated method, such tau hyperphosphorylation occurs via a TLR4-mediated immune response in a presently unknown mechanism that also involves the intermediacy of FNLA. For in vivo use, administration of a FLNA-binding benzazocine-ring compound is typically repeated a plurality of times and can be discontinued when a) the amount of tau protein phosphorylated at one or more of the three protein sequence positions (S²⁰², T²³¹, and T¹⁸¹) becomes constant, or b) until the amount of one or more of the TLR4 activation protein markers is at background levels, or c) both.

It is presently believed that each of the above pathways, Aβ-αnAChR and TLR4, can operate at the same time and also independently. Illustrative CNS conditions that exhibit one or both of Aβ-α7nAChR-mediated and/or TLR4-mediated tau phosphorylations of one or more of S²⁰², T²³¹ and T¹⁸¹ include those of persons and other animals whose CNS cells exhibit an immune response such as inflammation induced by brain injury such as traumatic brain injury (e.g., concussion), chronic traumatic encephalopathy, those having Alzheimer's disease (AD) symptoms, frontotemporal dementia (FTD), progressive supranuclear palsy, dementia pugilistica and corticobasal degeneration and also infection by one or both of Gram positive and Gram negative bacteria.

The binding of a SEQ ID NO: 1 FLNA pentapeptide by a contemplated FLNA-binding benzazocine-ring compound is determined as discussed in Example 1. A contemplated compound is substantially free from binding with any other portion of FLNA. Substantial freedom from binding with any other portion of FLNA can be determined using a titration assay such as that shown in FIG. 1 herein [FIG. 3 of Wang et al., PLoS One. 3(2):e1554 (2008)], which in that figure indicates the presence of two binding site regions by the two inflection points shown in the plot, whereas the presence of a single binding site is indicated by the presence of a single inflection point. Substantial freedom from binding with any other portion of FLNA can also be inferred from functional data such as a cytokine release assay illustrated hereinafter that indicates contemplated FLNA-binding benzazocine-ring compounds do not bind the second site on FLNA because the compounds are effective over a wide range of concentrations, unlike those compounds such as naloxone and naltrexone that bind two binding sites on FLNA.

In presently preferred embodiments, the present invention contemplates a method of inhibiting hyperphosphorylation of the tau protein at one or more of S²⁰², T²³¹ and T¹⁸¹ that comprises the step of administering to cells of the central nervous system in recognized (diagnosed) need such as brain cells of a patient suspected to have Alzheimer's disease or a tauopathy an effective amount of one or more FLNA-binding benzazocine-ring compounds such as a morphinan or 4,5-epoxymorphinan ring compound, a benzomorphan ring compound or a pharmaceutically acceptable salt thereof. The administration is often carried out a plurality of times.

The present invention has several benefits and advantages.

One benefit is that a contemplated method inhibits Aβ signaling through α7nAChR that is believed superior to targeting the receptor itself. Disabling the Aβ-induced α7nAChR signaling without directly affecting the α7nAChRs avoids altering the sensitivity or cell surface level of the receptors, an insidious problem with using chronic receptor agonists or antagonists.

An advantage of this invention is that this approach appears to selectively affect the robust increase in filamin recruitment by Aβ while preserving basal coupling, suggesting that a FLNA-binding benzazocine-ring compound used in the method reduces the pathological signaling by Aβ while retaining physiological α7nAChR signaling.

Another benefit of the invention is that administration of a contemplated FLNA-binding benzazocine-ring compound inhibits the in vitro and in vivo hyperphosphorylation of the tau protein.

Another advantage of the invention is that when a contemplated FLNA-binding benzazocine-ring compound is administered in vivo, the administration inhibits the formation of NFTs in the brain of a subject mammal to which a contemplated benzazocine-ring compound is administered.

Yet another benefit of the invention is that administration of a contemplated FLNA-binding benzazocine-ring compound can provide the benefits of one or more of the methods enumerated above by binding of that compound to the FLNA pentapeptide of SEQ ID NO: 1 disrupting one or more of the newly-found interactions of FLNA.

Yet another advantage of the invention is that its use can lessen the effects of tau hyperphosphorylation in persons or other animals with head injuries and resultant TLR4-mediated inflammation.

Still further benefits and advantages will be apparent to those skilled in the art from the disclosures that follow.

### BRIEF DESCRIPTION OF THE DRAWINGS

In the drawings forming a part of this disclosure,

FIG. 1, in two panels as FIG. 1A and FIG. 1B, is a graph that illustrates the binding of radio-labeled naloxone {[³H]NLX} to FLNA in the membranes of A7 cells in the presence of indicated, increasing amounts of naltrexone (NTX) (FIG. 1A) and is taken from Wang et al., PLoS One. 3(2):e1554 (2008), FIG. 3. FIG. 1B is a similar plot obtained from the use of FITC-labeled NLX (FITC-NLX) in place of the radio-labeled material.

FIG. 2, in two panels as FIG. 2A and FIG. 2B, illustrates high-affinity FLNA-binding compounds reduce α7nAChR-FLNA association as shown by Western blots. Frontal cortical synaptosomes from 2-month-old rats (n=4) were treated with 0.1 or 1 nM concentrations of potential FLNA-binding compounds [A0040, A0068, B0055, C0137M, C0138M and (+)-naloxone (+NLX)] either simultaneously (Sim) with or 10 minutes prior (10′ pr) to Aβ₄₂ and were analyzed for their α7nAChR-FLNA complex contents. The α7nAChR-FLNA complexes in the solubilized synaptosomes were immunoprecipitated with immobilized anti-FLNA and the α7nAChR and FLNA levels in the anti-FLNA immunoprecipitates determined by Western blotting FIG. 2A and quantified by densitometry (FIG. 2B). n=3. Data are means±SEM. **p<0.05, *p<0.01 vs. Aβ₄₂ alone. Data for 0.1 nM simultaneous admixture are shown in diagonal hatching; data from 1 nM simultaneous admixture are shown in cross-hatching; data from 0.1 nM 10 minutes prior to Aβ₄₂ are shown in horizontal hatching; and data from 1 nM admixture 10 minutes prior Aβ₄₂ are shown with vertical hatching. The letter designation “M” that accompanies many of the “C-series” compounds is omitted from FIG. 2, the remaining figures and most discussions of the figures and compounds hereinafter for ease in expression. Structural formulas of the compounds used in this and the other figures are provided hereinafter. The structures and syntheses of the numbered compounds in these figures can be found hereinafter as well as in one or more of in one or more of U.S. patent application Ser. No. 12/263,257, Ser. No. 12/435,284, Ser. No. 12/607,883, Ser. No. 12/435,355, Ser. No. 12/719,589, and Ser. No. 12/719,624, whose disclosures are incorporated by reference.

FIG. 3, in two panels as FIG. 3A and FIG. 3B, illustrates that FLNA-binding compounds reduce Aβ-induced α7nAChR-mediated ERK2 signaling. In the same treated synaptosomes used in FIG. 2, levels of phosphorylated (activated) ERK2 (pERK2) were measured in immunoprecipitates of ERK2. Aβ₄₂ strongly activated extracellular signal-regulated kinase 2 [ERK2; also known as mitogen-activated protein kinase 1 (MAPK1)], and all compounds studied reduced this activation with 10-minutes of pretreatment. Two compounds, B0055 and (+)-naloxone (FIG. 3B) reduced the activation at both concentrations when provided simultaneously. Immunoprecipitates were determined by Western blotting (FIG. 3A) and quantified by densitometry (FIG. 3B). n=3. Data are means±SEM. **p<0.05, *p<0.01 vs. Aβ₄₂ alone. Hatching is as in FIG. 2.

FIG. 4, in four panels as FIG. 4A, FIG. 4B, FIG. 4C and FIG. 4D, illustrate that FLNA-binding compounds reduce tau phosphorylation at all three hyperphosphorylation sites. In the same treated synaptosomes used in FIGS. 2 and 3, levels of tau protein phosphorylated at S²⁰², T²³¹ and T¹⁸¹ were measured in immunoprecipitates using an anti-tau antibody that does not distinguish its phosphorylation state. The three phosphoepitopes of tau were detected in immunoprecipitates using specific antibodies in Western blots. Aβ₄₂ strongly promotes tau phosphorylation at all three sites, and all compounds reduced this phosphorylation with 10-minute pretreatment. Each assayed compound, including (+)-naloxone, reduced tau phosphorylation with simultaneous administration at both concentrations studied. Immunoprecipitates were determined by Western blotting (FIG. 4A) and quantified by densitometry (FIG. 4B, FIG. 4C and FIG. 4D). Data are means±SEM. **p<0.05, *p<0.01 vs. Aβ₄₂ alone. Hatching is as in FIG. 2.

FIG. 5 through FIG. 10 represent schematic pharmacophores (Pharmacophores 1-6, respectively) showing relative locations of chemical features such as a hydrogen bond acceptor (HBA), an aromatic/hydrophobe (ARO/HYD) center, and the intramolecular distances there between in Ångstroms for a compound that binds to the pentameric peptide of FLNA of SEQ ID NO: 1.

FIG. 11 is a graph that compares the inhibition of 10 nM FITC-conjugated NLX binding to the biotinylated FLNA pentapeptide of SEQ ID NO: 1 using NLX, NTX, morphine and oxycodone at the indicated concentrations.

### ABBREVIATIONS AND SHORT FORMS

The following abbreviations and short forms are used in this specification.

“Aβ” means amyloid-beta

“Aβ₄₂” means a 42-residue proteolysis product of amyloid precursor protein (APP)

“α7nAchR” means alpha-7 nicotinic acetylcholine receptor

“DAMGO” means [D-Ala2, N-MePhe4, Gly-ol]-enkephalin

“ERK2” means extracellular signal-regulated kinase 2

“FCX” means frontal cortex or prefrontal cortex

“FLNA” means filamin A

“FITC” means fluorescein isothiocyanate

“Gs” means G protein stimulatory subtype, stimulates adenylyl cyclase

“HP” means hippocampus

“IHC” means immunohistochemistry

“IR” means insulin receptor

“MOR” means p opioid receptor

“NLX” means naloxone

“NTX” means naltrexone

“NFTs” means neurofibrillary tangles

“NMDA” means N-methyl-D-aspartate

“NMDAR” means NMDA receptor

“pERK2” means phosphorylated ERK2

“pTau” means hyperphosphorylated tau protein

“TLR4” means toll-like receptor-4

### DEFINITIONS

In the context of the present invention and the associated claims, the following terms have the following meanings:

The articles “a” and “an” are used herein to refer to one or to more than one (i.e., to at least one) of the grammatical object of the article. By way of example, “an element” means one element or more than one element.

As used herein, the term “binds” refers to the specific adherence of molecules to one another, such as, but not limited to, the interaction of a ligand with its receptor, or a polypeptide of SEQ ID NO: 1 with a small molecule such as the compounds disclosed herein, or an antibody and its antigen.

As used herein, the term “FLNA-binding benzazocine-ring compound” refers to a compound that contains such a ring structure and binds to the scaffolding protein filamin A, or more preferably to a polypeptide comprising residues -Val-Ala-Lys-Gly-Leu- (SEQ ID NO: 1) of the FLNA sequence that correspond to amino acid residue positions 2561-2565 of the FLNA protein sequence as noted in the sequence provided at the web address: UniProtKB/Swiss-Prot entry P21333, FLNA-HUMAN, Filamin-A protein sequence. A FLNA-binding benzazocine-ring compound can inhibit the MOR-Gs coupling caused by agonist stimulation of the μ opioid receptor via interactions with filamin A, preferably in the 24^{th} repeat region. A FLNA-binding benzazocine-ring compound binds to the FLNA sequence of the pentapeptide of SEQ ID NO: 1 with an affinity that is about 100- to about 15,000-times greater than to any other sequence within the FLNA protein, and can be said to bind specifically to the pentapeptide sequence of SEQ ID NO: 1.

As used herein an “FLNA-binding effective amount” or more simply an “effective amount” refers to an amount of a contemplated FLNA-binding benzazocine-ring compound sufficient to bind to the FLNA pentapeptide of SEQ ID NO: 1 and perform the functions described herein, such as inhibition of tau protein phosphorylation. An effective amount of a contemplated FLNA-binding benzazocine-ring compound is most easily determined using the in vitro assay of Example 1. Using that definition, an effective amount of a contemplated FLNA-binding benzazocine-ring compound binds to a pentapeptide of SEQ ID NO: 1, inhibits at least about 60 percent and more preferably about 70 percent of the FITC-labeled naloxone binding when present at a 10 μM concentration and using unlabeled naloxone as the control inhibitor at the same concentration and under the same conditions as the contemplated compound, and up to about twice (200 percent) the inhibition obtained with naloxone as control.

As used herein, the term “opioid receptor” refers to a G protein-coupled receptor located in the CNS that interacts with opioids. More specifically, the μ opioid receptor is activated by morphine causing analgesia, sedation, nausea, and many other side effects known to one of ordinary skill in the art. The μ opioid receptor is the receptor usually involved in the carrying out of this invention, although the κ-opioid (kappa-opioid) receptor and δ-opioid receptor (delta-opioid receptor) are also sometimes involved.

As used herein, the term “opioid agonist” refers to a substance that upon binding to an opioid receptor can stimulate the receptor, induce G protein coupling and trigger a physiological response. More specifically, an opioid agonist is a morphine-like substance that interacts with MOR to produce analgesia.

As used herein, the term “opioid antagonist” refers to a substance that upon binding to an opioid receptor inhibits the function of an opioid agonist by interfering with the binding of the opioid agonist to the receptor.

As used herein the term “pharmacophore” is not meant to imply any pharmacological activity. A pharmacophore can be defined as the relevant groups on a molecule that interact with a receptor and are responsible for the activity of the compound. [R. B. Silverman, The Organic Chemistry of Drug Design and Drug Action, 2^{nd} ed., Elsevier Academic Press, Amsterdam, (2004), p. 17.] The term can also be defined and is intended herein to be the chemical features of a molecule and their distribution in three-dimensional space that constitutes the preferred requirements for molecular interaction with a receptor (See, U.S. Pat. No. 6,034,066). A pharmacophore is computer-calculated by determining the shared aromatic/hydrophobic and hydrogen bond acceptor functions and the distances there between of a group of compounds that bind similarly to a particular receptor, here, pentapeptide of SEQ ID NO: 1, using an appropriately programmed computer. Such computer programs are available commercially from companies such as Accelrys Software Inc., San Diego, Calif., Schrodinger, LLC, Portland, Oreg., from Chemical Computing Group, Inc., Montreal, QC, Canada, or as an open access program referred to as ZINCPharmer

### DETAILED DESCRIPTION OF THE INVENTION

The present invention contemplates a method of inhibiting hyperphosphorylation (phosphorylation) of the tau protein at one or more of S²⁰², T²³¹ and T¹⁸¹ in vitro as well as in vivo. That method comprises the steps of administering to central nervous system cells in recognized (diagnosed) need thereof, such as brain cells, an FLNA-binding effective amount of a FLNA-binding benzazocine-ring compound or a pharmaceutically acceptable salt thereof such as a) an opioid receptor antagonist compound, b) a mixed opioid receptor agonist and antagonist (agonist/antagonist) compound, c) an opioid receptor agonist compound or d) an enantiomer of an opioid receptor interacting compound (i.e., antagonist, agonist/antagonist or agonist compound). A contemplated FLNA-binding benzazocine-ring compound binds to filamin A or binds to a pentapeptide of filamin A (FLNA) of SEQ ID NO: 1 as described in Example 1, and inhibits at least about 60 percent and more preferably about 70 percent of the FITC-labeled naloxone binding to FLNA or that FLNA pentapeptide when present at a 10 μM concentration and using unlabeled naloxone as the control inhibitor at the same concentration.

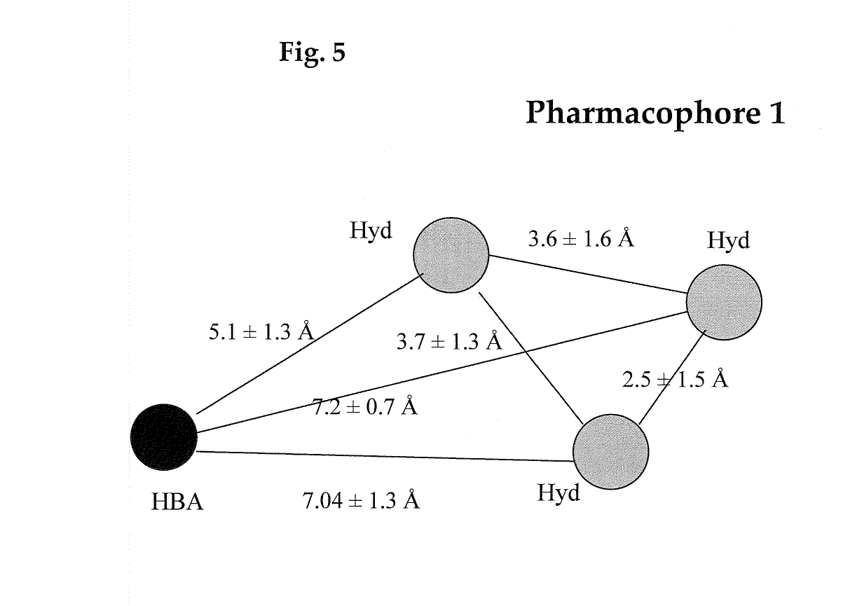

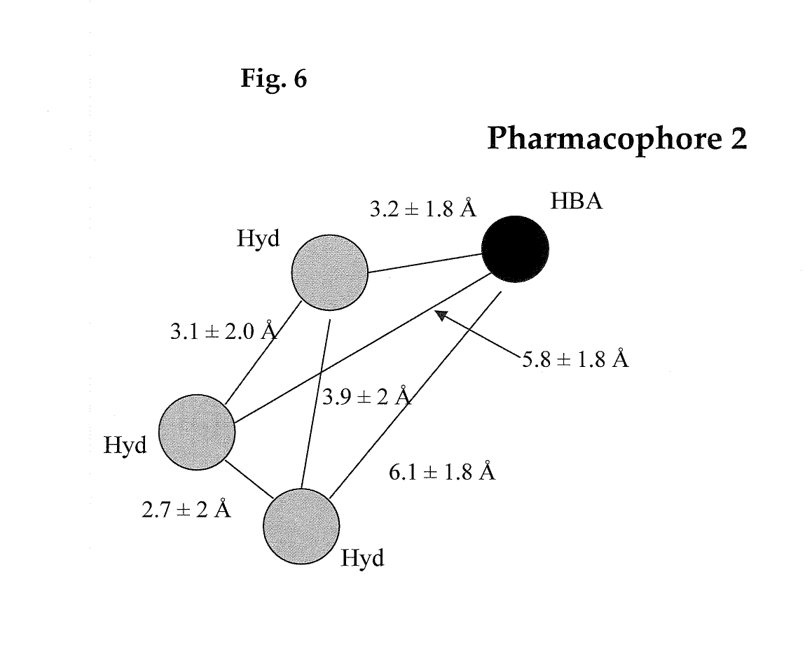

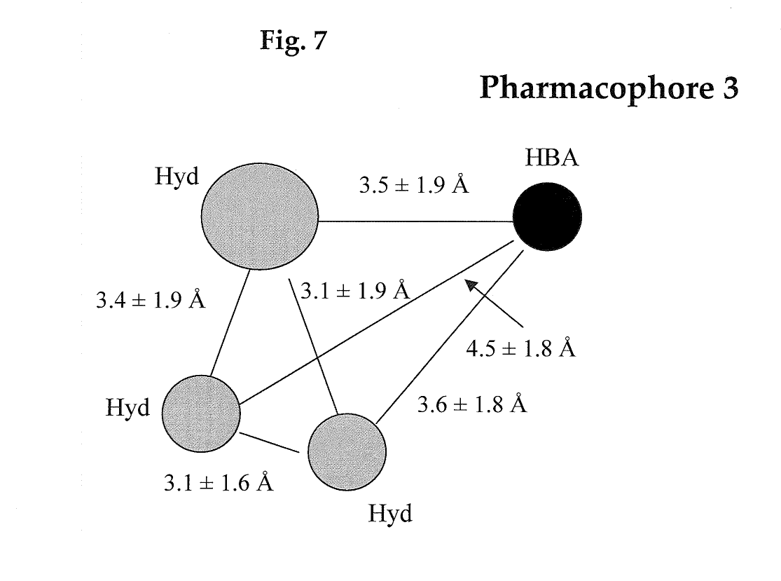

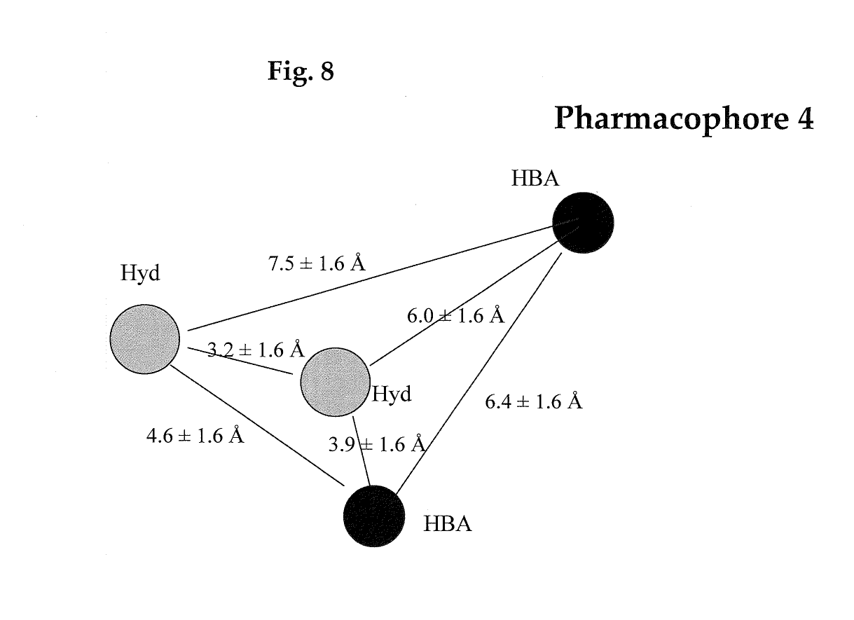

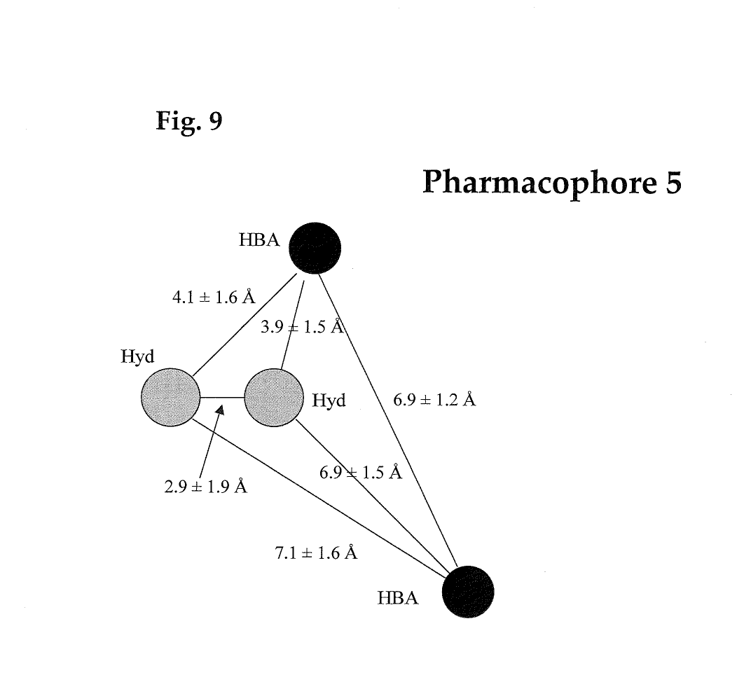

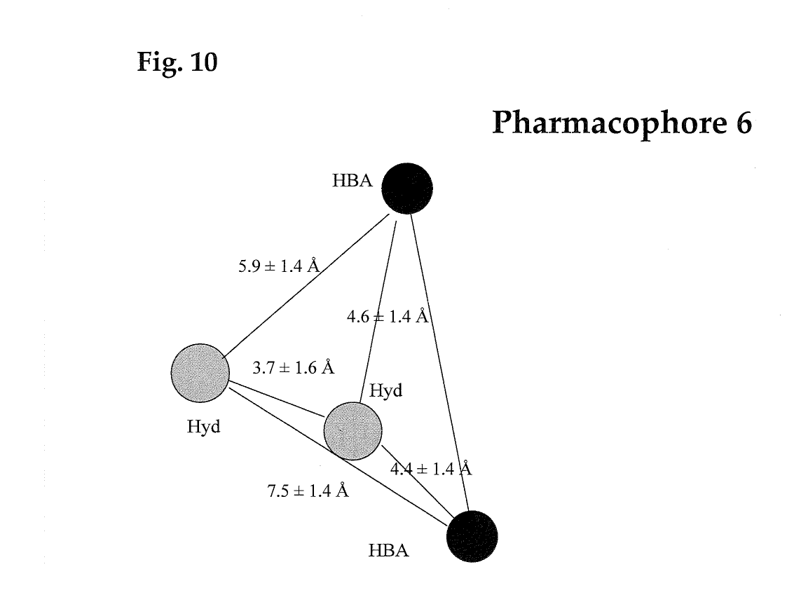

That FLNA-binding benzazocine-ring compound also preferably contains at least four of the six pharmacophores of FIGS. 5-10. A more preferred FLNA-binding benzazocine-ring compound contains five of the six pharmacophores, and a most preferred FLNA-binding benzazocine-ring compound contains all six of those pharmacophores. An illustrative benzazocine-ring compound includes a morphinan, 4,5-epoxymorphinan ring compound as well as a benzomorphan ring compound, as are discussed hereinafter.

The administration is preferably carried out in the absence of a mu opioid receptor (MOR)-binding effective amount of a separate MOR agonist or antagonist molecule. Administration of a contemplated FLNA-binding benzazocine-ring compound or its pharmaceutically acceptable salt in vivo is typically repeated a plurality of times. That in vivo administration is continued until tau hyperphosphorylation at one or more of the three protein sequence positions (S²⁰², T²³¹, and T¹⁸¹) becomes constant. Thus, after injury, tau phosphorylation at one or more of the above-noted protein positions increases, and although a contemplated administration may not reverse that reaction, increased hyperphosphorylation can be inhibited so that the amount of hyperphosphorylated protein plateaus to a constant level (within measuring limits).

Phosphorylation of one or more of S²⁰², T²³¹ and T¹⁸¹ of the tau protein is typically in addition to phosphorylation of one or more additional residues of the protein. The presence of such hyperphosphorylation can be determined by the immunoreaction of an antibody, usually a monoclonal antibody, that immunoreacts specifically with a tau protein that is phosphorylated at one of those three amino acid residues as is illustrated herein.

Also contemplated is a method of inhibiting TLR4-mediated immune response such as inflammation of TLR4-containing cells such as lymphocytes or cells of the CNS. That method comprises administering to TLR4-containing cells in recognized need thereof a FLNA-binding effective amount of a FLNA-binding benzazocine-ring compound or a pharmaceutically acceptable salt thereof such as a) an opioid receptor antagonist compound, b) a mixed opioid receptor agonist and antagonist (agonist/antagonist), c) an opioid receptor agonist compound or d) an enantiomer of an opioid receptor interacting compound (i.e., antagonist, agonist/antagonist or agonist compound), that binds to a pentapeptide of filamin A (FLNA) of SEQ ID NO: 1. A contemplated FLNA-binding benzazocine-ring compound inhibits at least about 60 percent and more preferably about 70 percent of the FITC-labeled naloxone binding to the FLNA pentapeptide of SEQ ID NO: 1 when present at a 10 μM concentration and using unlabeled naloxone as the control inhibitor at the same concentration.

A contemplated FLNA-binding benzazocine-ring compound preferably contains at least four of the six pharmacophores of FIGS. 5-10. A more preferred FLNA-binding benzazocine-ring compound contains five of the six pharmacophores, and a most preferred FLNA-binding benzazocine-ring compound contains all six of those pharmacophores. The administration is preferably carried out in the absence of a mu opioid receptor (MOR)-binding effective amount of a separate MOR agonist or antagonist molecule.

TLR4-mediated immune response inflammation of CNS cells produces hyperphosphorylation of the tau protein and related tauopathies such as those that result from NFTs. As a consequence, one way to assay for the presence of TLR4-mediated inflammation is to assay for the presence of an enhanced amount of phosphorylated tau compared to the amount present in a non-inflammatory condition as was described above for hyperphosphorylated tau.

TLR4-mediated inflammation can also be recognized by the greater than background abundance of TLR4 activation protein markers such as the cytokines IL-1β, IL-6 and TNFα that are typically enhanced together, and/or the separately stimulated NF-κB and JNK proteins. As was noted earlier, enhanced expression of IL-1β, IL-6 and TNFα as compared to expression of NF-κB and JNK appear to proceed by different TLR4-mediated pathways. However, both markers of inflammation can be present at the same time due to the same immunostimulus.

Thus, the presence of an enhanced amount of one, two or three of IL-1β, IL-6 and TNFα relative to the amount present in a non-inflammatory condition indicates the presence of TLR4-mediated inflammation. Similarly, the enhanced presence of the transcription factor NF-κB and the mitogen-activated protein kinase c-Jun N-terminal kinase (JNK) compared to the amount present in a non-inflammatory condition separately implies the presence of TLR4-mediated inflammation.

These proteins or polypeptides (e.g., IL-1β, IL-6, TNFα, NF-κB and JNK) can be assayed in lysates of cultured cells such as lymphocytes such as B cells, T cells and macrophages or CNS cells such as olfactory neurons that can be obtained by scraping the nasal cavity for neural epithelial cells for in vivo assays. The proteins can also be assayed in the cell culture medium for in vitro studies using lymphocytes or CNS cells such as those illustrated hereinafter and in body fluids such as blood or its constituent plasma or serum or lymphocytes for in vivo assays.

Administration of a contemplated FLNA-binding benzazocine-ring compound or its pharmaceutically acceptable salt in vivo is typically repeated a plurality of times. That in vivo administration is continued until tau hyperphosphorylation at one or more of the three protein sequence positions (S²⁰², T²³¹, and T¹⁸¹) becomes constant, as discussed above, and/or until the amount of one or more of the TLR4 activation protein markers returns to background levels.

Enhancement of the level of hyperphosphorylated tau or one or more of the TLR4 protein markers relative to background (in the absence of a TLR4-mediated immune response) condition can be determined by a difference that is statistically significant at least at the 90 percent confidence level (p<0.1), and preferably at the 95 percent confidence level (p<0.05) as are illustrated in the accompanying figures.

It is also preferred that a FLNA-binding benzazocine-ring compound or a pharmaceutically acceptable salt thereof be present dissolved or dispersed in a pharmaceutically acceptable diluent as a pharmaceutical composition when administered. Most preferably, the administration is peroral, although parenteral administration as by subcutaneous, intravenous, intramuscular, intrasternal injection, or infusion techniques can also be used.

A preferred FLNA-binding benzazocine-ring compound or salt contemplated for use in a contemplated method is often commercially available dissolved or dispersed in a pharmaceutically acceptable diluent. That commercial pharmaceutical composition can be diluted for use herein to provide a desired concentration as discussed hereinafter, or reformulated at an appropriate concentration using a pharmaceutically acceptable diluent similar to that of a commercial product that contains a FLNA-binding benzazocine-ring compound or salt that is utilized.

Illustrative commercially available pharmaceutical compositions containing a contemplated preferred FLNA-binding benzazocine-ring compound or salt can be found in the Physicians' Desk Reference, Medical Economics Co., Montvale, N.J. Formulation of compounds similar to a FLNA-binding benzazocine-ring compound or salt useful in the present invention is discussed in, for example, Hoover, John E., Remington's Pharmaceutical Sciences, Mack Publishing Co., Easton, Pa. 1975; and Liberman, H. A. and Lachman, L., Eds., Pharmaceutical Dosage Forms, Marcel Decker, New York, N.Y., 1980.

The use of a pharmaceutically acceptable salt of a contemplated FLNA-binding benzazocine-ring compound is also contemplated, as is the use of a single stereoisomer or mixture of stereoisomers, or of their pharmaceutically acceptable salts. The reader is directed to Berge, 1977 J. Pharm. Sci. 68(1):1-19 for lists of commonly used pharmaceutically acceptable acids and bases that form pharmaceutically acceptable salts with pharmaceutical compounds. The contemplated administration can take place in vivo or in vitro.

In presently preferred embodiments, the present invention contemplates a method of inhibiting 1) hyperphosphorylation of the tau protein and/or 2) TLR4-mediated immune response (e.g., inflammation) of lymphocytes and/or cells of the CNS that comprises administering to cells of the central nervous system in recognized need thereof such as brain cells an effective amount of a FLNA-binding benzazocine-ring compound or salt discussed herein below. A single enantiomer, a mixture of enantiomers or a pharmaceutically acceptable salt of any contemplated FLNA-binding benzazocine-ring compound(s) can be used. The administration is preferably carried out in the absence of a MOR-binding effective amount of a separate MOR agonist or antagonist molecule.

Illustrative of CNS cells are cells such as those of a host animal that exhibit inflammation induced by brain injury such as traumatic brain injury, chronic traumatic encephalopathy, as well as those of a host animal such as a human exhibiting Alzheimer's disease (AD) symptoms, frontotemporal dementia (FTD), progressive supranuclear palsy, dementia pugilistica and corticobasal degeneration, as well as infection by Gram positive and/or Gram negative bacteria.

In accordance with a method described above, a composition that contains an effective amount of a contemplated FLNA-binding benzazocine-ring compound or its pharmaceutically acceptable salt dissolved or dispersed in a pharmaceutically acceptable diluent is administered to cells of the CNS in recognized need thereof, and particularly the brain, in vivo in a living animal or to cells in vitro in a cell preparation. When administered in vivo to an animal such as a laboratory rat or mouse or a human in recognized need, the administration inhibits the formation of phosphorylated-tau-containing NFTs in the CNS such as in the brain of a subject animal to which a contemplated compound is administered. Admixture of a composition containing a contemplated compound or its pharmaceutically acceptable salt dissolved or dispersed in a pharmaceutically acceptable diluent with CNS cells such as a brain cell preparation in vitro also inhibits the formation of NFTs.

A contemplated FLNA-binding benzazocine-ring compound binds to the scaffolding FLNA protein, and particularly to a five residue portion of the FLNA protein sequence—Val-Ala-Lys-Gly-Leu (SEQ ID NO: 1)—in an in vitro assay that is discussed hereinafter in Example 1, and briefly below. A contemplated FLNA-binding benzazocine-ring compound binds only to a single site on FLNA and that site contains the amino acid residue sequence of the SEQ ID NO: 1 pentapeptide.

Binding studies of the naltrexone inhibition of tritiated-naloxone, [³H]NLX, binding to membranes from FLNA-expressing A7 cells (an astrocyte cell line produced by immortalizing optic nerve astrocytes from the embryonic Sprague-Dawley rat with SV40 large T antigen) has shown the existence of two affinity sites on FLNA; a high affinity site (H) with an IC₅₀-H of 3.94 picomolar and a lower affinity site (L) IC₅₀-L of 834 picomolar. [Wang et al., PLoS One. 3(2):e1554 (2008); Wang et al., PLoS One. 4(1):e4282 (2009).] The high affinity site was subsequently identified as the FLNA pentapeptide of SEQ ID NO: 1 (US Patent Publication 2009/0191579 and its predecessor application Ser. No. 60/985,086 that was filed on Nov. 2, 2007), whereas the lower affinity site has not yet been identified.

FLNA-binding benzazocine-ring compounds such as naloxone (NLX), naltrexone (NTX), nalorphine, nalbuphine and buprenorphine, and the like bind well to the high affinity FLNA pentapeptide of SEQ ID NO: 1 (VAKGL). However, when used at a dosage recited on the label of one of those drug products, those compounds also bind to the lower affinity site on FLNA, and some also bind to the mu opiate receptor. Some of the compounds are MOR antagonists such as naloxone, naltrexone, nalbuphine, whereas others such as buprenorphine and etorphine are full or partial agonists of MOR. (+)-Naloxone is the inactive enantiomer of (−)-naloxone that is active at (interacts with) MOR, and therefore (+)NLX is neither an antagonist nor agonist.

Binding to that lower affinity FLNA site as well as to the high affinity site of FLNA can impair the activity of FLNA to exhibit its activities as discussed, utilized and illustrated herein. As a consequence, FLNA-binding benzazocine-ring compounds such as naloxone, naltrexone, nalorphine, nalbuphine, buprenorphine and the other FLNA-binding benzazocine-ring compounds contemplated herein that also bind to the lower affinity site on the FLNA protein are not contemplated for use at the dosages indicated on their product labels. Rather, a contemplated FLNA-binding benzazocine-ring compound is utilized at a level that is about 1000^{th} to about 1,000,000^{th} the minimal dosage listed on the product labels (as found in the Physicians' Desk Reference, Medical Economics Co., Montvale, N.J.), and more preferably at a level that is about 10,000^{th} to about 100,000^{th} the dosage listed on the product labels.

In one embodiment, it is preferred to use a FLNA-binding benzazocine-ring compound or pharmaceutically acceptable salt of other than naltrexone or naloxone. In another embodiment, naltrexone or naloxone or its salt is a preferred FLNA-binding benzazocine-ring compound.

A similar binding assay to that discussed above using [³H]NLX was also be carried out using a different label linked to NLX. One such different, useful labeled compound is fluorescein isothiocyanate-labeled naloxone (FITC-NLX). The use of radioactive materials is also avoided with the use of FITC-NLX.

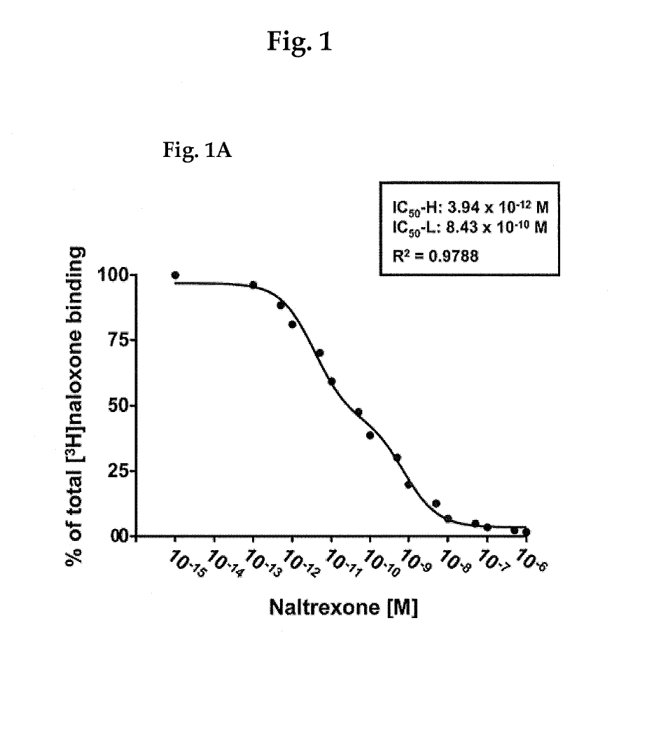

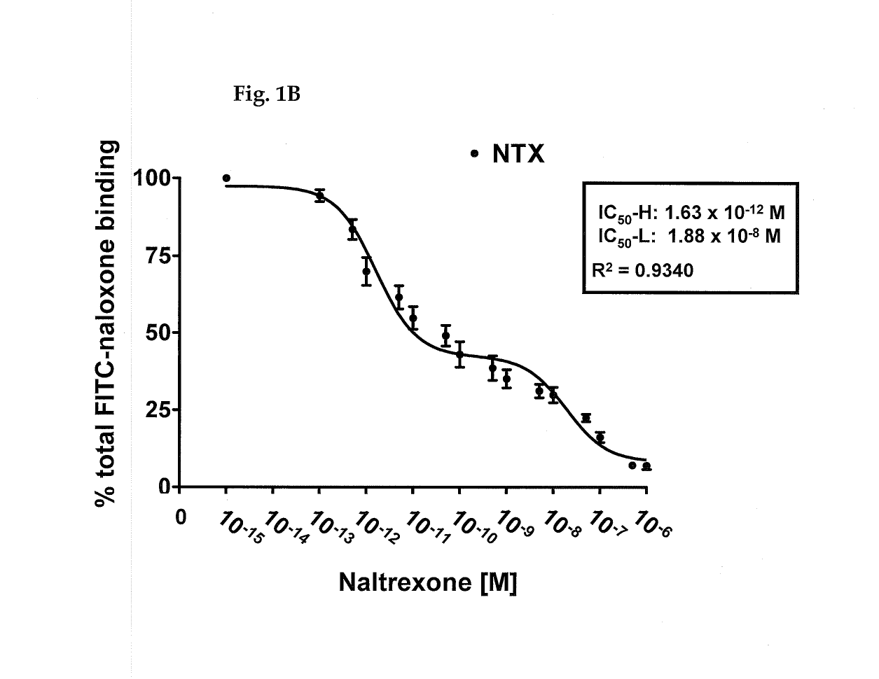

The results of binding inhibition studies obtained using FITC-NLX are very similar to those obtained with [³H]NLX, with affinities determined using FITC sometimes being of somewhat lesser magnitude, likely due to steric factors caused by the fluorescein-containing portion of the molecule. For example, the IC_{50-L} values obtained from the binding studies shown in FIG. 1A and FIG. 1B imply that the low affinity site of FLNA is quite sterically sensitive, whereas the high affinity site is not as sensitive. The results obtained for a given group of FLNA-binding benzazocine-ring compounds using FITC-NLX exhibit the same order of affinities as those obtained using tritium as the label. That is, if the order of affinities obtained using a tritium label were compound A>compound B>compound C, that same order of affinities is obtained using the FITC label. It is also more convenient to use the biotinylated high affinity FLNA pentapeptide of SEQ ID NO: 1 (Bn-VAKGL) rather than the full protein from a cell membrane preparation for the binding inhibition assay.

A FLNA-binding benzazocine-ring compound contemplated for use in the present invention inhibits the binding of FITC-NLX to biotin-linked SEQ ID NO: 1 (Bn-VAKGL) bound to coated streptavidin plates under conditions defined hereinafter in Example 1 to an extent that is at least about 60 percent and more preferably at least about 70 percent of the value obtained of the value obtained when present at a 10 μM concentration and using naloxone as the control inhibitor at the same concentration as the contemplated compound, and up to about twice the value obtained with naloxone as control.

Naltrexone (NTX) can also be used as a control inhibitor. Average inhibition values obtained using NTX rather than NLX tend to be 1 or 2 percentage digits lower in absolute value than those obtained with NLX. Thus, for example, where an average inhibition value at a particular concentration of NLX is 40 percent, one can expect values obtained with NTX to be about 38 or 39 percent. The binding inhibition values for a contemplated FLNA-binding benzazocine-ring compound are determined taking the expected NLX/NTX value difference into account.

### Pharmacophore Determinations

One aspect of the invention is the use of a FLNA-binding benzazocine-ring compound that binds to the FLNA pentapeptide of SEQ ID NO: 1 to inhibit hyperphosphorylation of the tau protein. In this aspect, the structures of the compounds that effectively bind to a pentapeptide of SEQ ID NO: 1 are similar and can be unified through the computer-assisted calculation of a group of pharmacophores shared by those compounds that so bind.

A contemplated FLNA-binding benzazocine-ring compound useful in a method of the invention preferably contains at least four of the six pharmacophores of FIGS. 5-10. In more preferred practice, a contemplated compound contains five of the six pharmacophores of those figures, and most preferably, a contemplated FLNA-binding benzazocine-ring compound contains all six of the pharmacophores.

An ensemble pharmacophore model was prepared with a programmed general purpose computer using the three-dimensional conformations of compounds in the training sets. Using 0.1 μM data from Example 2 of each of WO 2010/051497 A1, WO 2010/051374 A1 and WO 051476 A1 that were all published on May 6, 2010 and Example 2 of US Patent Publication No. 2010/0280061 A1 published on Nov. 4, 2010 as a starting point, 153 compounds out of the list of compounds in the tables of those Examples 2 have a binding activity to the FLNA pentapeptide that is less than the mean value of 45.54 percent. A “poor binding” compound or “poor binder” is defined as a compound whose binding inhibition is equal to or less than the mean value of 45.54 percent in an assay as conducted in Example 1 herein. The training set consists of ten compounds known to bind to the FLNA pentapeptide, the above poor binding 153 compounds and also about 1000 random compounds selected from ZINC database at zinc.docking.org.

The selection of pharmacophores involves in the following steps: 1) Three-dimensional computer-generated conformations of all FLNA-binding benzazocine-ring compounds were first prepared. 2) A set of 4-point pharmacophores present in most of known active compounds was derived. 3) Using known inactive and random selected compounds as reference compounds, only those pharmacophores that were not present in the most of the reference compounds were identified as relevant to FLNA binding activity. 4) Six 4-point pharmacophores were finally identified from those determined above to best represent the 10 active compounds.

An untested compound that contains four out of the six pharmacophores has about a 20 percent chance to be an active binder to the FLNA pentapeptide. A compound containing five of the six pharmacophores has about a 32 percent chance to be an active binder to the FLNA pentapeptide, and about a 60 percent chance when containing six of the six pharmacophores.

The Molecular Operating Environment (MOE) software from Chemical Computing Group, Montreal, Quebec, Canada, was used to program a general purpose computer to generate three-dimensional conformations, to derive 4-point pharmacophores from active FLNA-binding benzazocine-ring compounds, and to test these pharmacophores against known inactive compounds and random selected compounds. Pharmacophore modeling as used herein is carried out as discussed and explained in Penzotti et al., J Med Chem, 2002, 45(9):1737-1740 (2002); Siew et al., Bioorg Med Chem Lett, 21(10):2898-2905 (15 May 2011); Leong, Chem Res Toxicol, 20(2):217-226 (2007); and Lin, chemcomp.com/journal/ph4.htm.

In some embodiments, it is preferred that a FLNA-binding benzazocine-ring compound also be a MOR agonist. In other embodiments, it is preferred that the FLNA-binding benzazocine-ring compound not be a MOR agonist. A compound is defined herein as not being a MOR agonist if it has less than about 80 percent the binding of [D-Ala2,N-MePhe4,Gly-ol]-enkephalin (DAMGO) at either of the two concentrations used in the Tables of Examples 1 of those published applications.

The ten known FLNA pentapeptide-binding training set compounds are shown below along with their alpha-numeric designations used herein. Of the

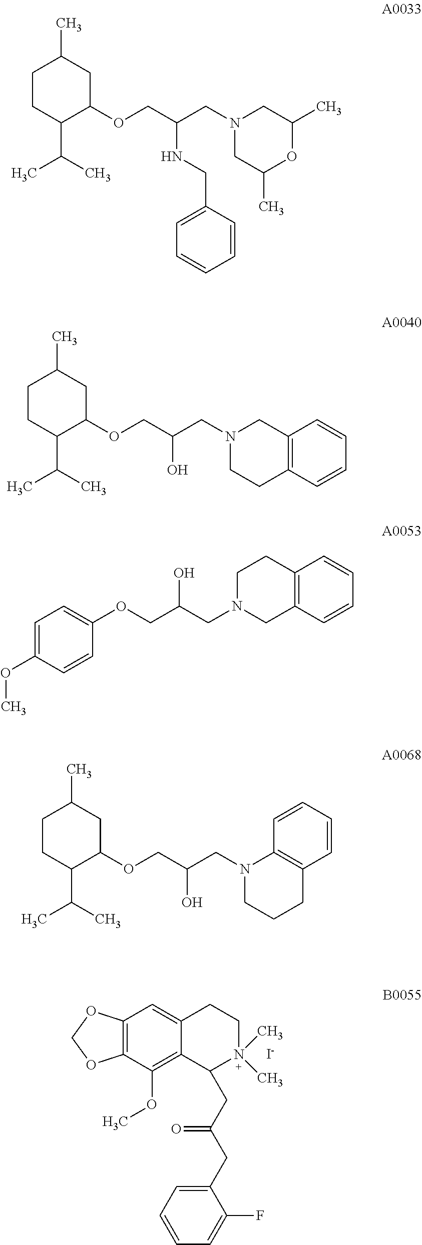

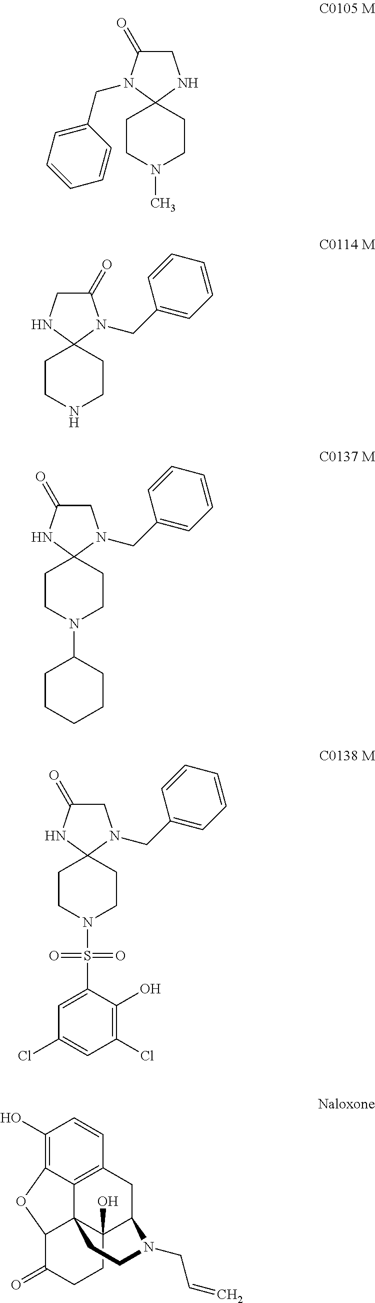

above ten compounds used in the training set for determining the pharmacophores, nine contained all six pharmacophores. Naloxone contained five of the six.

### Specifically Contemplated FLNA-Binding Benzazocine-Ring Compounds

A contemplated benzazocine-ring compound is typically a) an opioid receptor antagonist, b) a mixed opioid receptor agonist/antagonist compound, c) an opioid receptor agonist compound or d) an enantiomer of an opioid receptor interacting compound. A contemplated FLNA-binding benzazocine-ring compound binds to a FLNA pentapeptide of SEQ ID NO: 1 as described in Example 1, preferably contains four, five or six of the pharmacophores of FIGS. 5-10, and is preferably a reversible antagonist at an opioid receptor.

One group of preferred FLNA-binding benzazocine-ring compounds is a morphinan or 4,5-epoxymorphinan ring compound. Morphinan ring compounds are analogs of morphine. The structural formulas of morphine and typical morphinan and 4,5-epoxymorphinan ring compounds are shown below, where R¹ and R² are substituent groups.

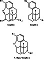

Preferably, the opioid receptor antagonized is at least the mu opioid receptor (MOR). Many of the morphinan or 4,5-epoxymorphinan ring compounds are analgesics that bind to the mu opioid receptor (MOR). The table below lists several benzazocine-ring compounds, the opioid receptor that they antagonize, and the relative potency of the antagonism or agonism as reported in Goodman & Gilman's The Pharmaceutical Basis of Therapeutics, 12^{th} ed., Brunton ed., McGraw-Hill Medical Publishing Division, New York, Table 18-1-3, pages 483-484 (2011). A morphinan or 4,5-epoxymorphinan ring compound is particularly preferred for use in a contemplated method. Of this group of particularly preferred compounds, a morphinan and 4,5-epoxymorphinan ring compound that is an opioid receptor antagonist is usually more particularly preferred.

| | RECEPTOR* | | |
|---|---|---|---|
| OPIOID LIGAND | MU | DELTA | KAPPA |
| Naloxone | −−− | − | −− |
| Naltrexone | −−− | − | −−− |
| Diprenorphine | −−− | −− | −−− |
| Naloxonazine | −−− | − | − |
| nor-Binaltrophimine | − | − | −−− |
| Naltrindole | − | −−− | − |
| Naloxone | −−− | − | − |
| Benzoylhydrazone | | | |
| Nalbuphine | −− | NR | ++ |
| Buprenorphine | P | NR | −− |
| Butorphanol | P | NR | +++ |
| Ethylketocyclazcine | P | + | +++ |
| Nalorphine | −−− | NR | + |

*“−” = antagonist;
“+” = agonist;
“NR” = not reported in the published table;
P = partial agonist;
The number of symbols in an indication of relative potency.

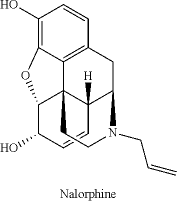 [SMILES: C=CCN1CC[C@]23c4c5ccc(O)c4O[C@H]2[C@@H](O)C=C[C@H]3[C@H]1C5]

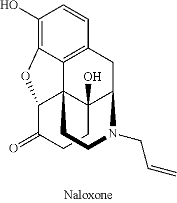

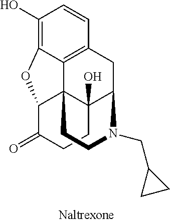

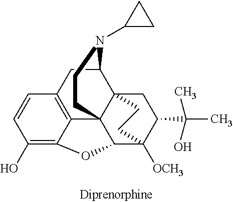

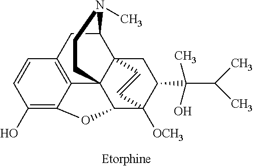

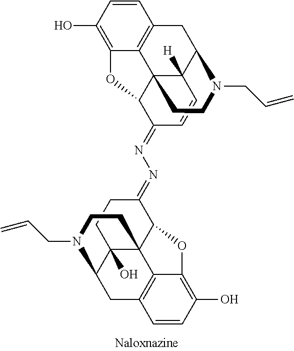

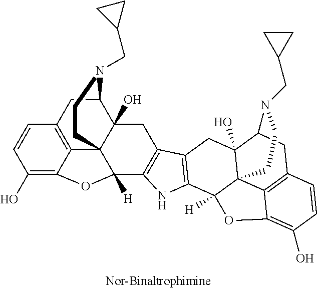

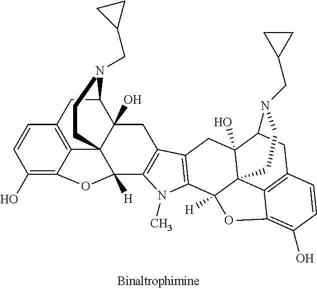

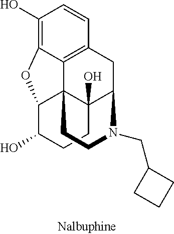

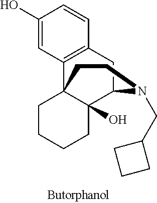

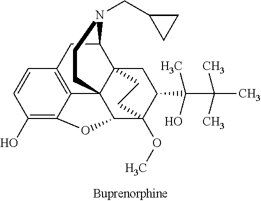

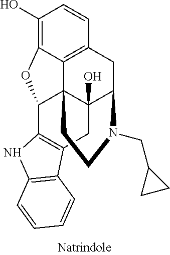

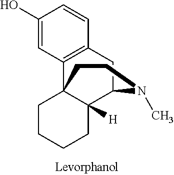

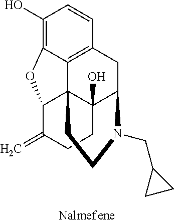

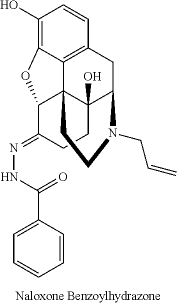

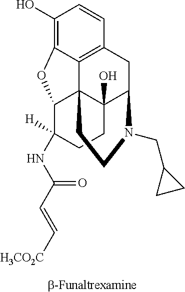

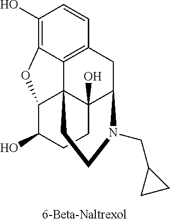

In accordance with a method described above, a composition that contains an effective amount of a contemplated FLNA-binding benzazocine-ring compound or its pharmaceutically acceptable salt dissolved or dispersed in a pharmaceutically acceptable diluent is administered to cells of the CNS in recognized need thereof, and particularly the brain, in vivo in a living animal or in vitro in a cell preparation. When administered in vivo to an animal such as a laboratory rat or mouse or a human in recognized need, the administration inhibits the formation of phosphorylated-tau-containing NFTs in the CNS such as in the brain of a subject animal to which a contemplated compound is administered. Admixture of a composition containing a contemplated compound or its pharmaceutically acceptable salt dissolved or dispersed in a pharmaceutically acceptable diluent with CNS cells such as a brain cell preparation in vitro also inhibits the formation of NFTs as is illustrated hereinafter.

A contemplated compound binds to the scaffolding protein FLNA, and particularly to a five residue portion of the FLNA protein sequence Val-Ala-Lys-Gly-Leu (SEQ ID NO: 1) in an in vitro assay that is discussed hereinafter in Example 1, and briefly below. A contemplated FLNA-binding benzazocine-ring compound binds only to a single site on the FLNA protein, and that site contains the pentapeptide of SEQ ID NO: 1.

Studies of the naltrexone inhibition of tritiated-naloxone, [³H]NLX, binding to membranes from FLNA-expressing A7 cells (an astrocyte cell line produced by immortalizing optic nerve astrocytes from the embryonic Sprague-Dawley rat with SV40 large T antigen) have shown the existence of two affinity sites on FLNA; a high affinity site (H) with an IC₅₀—H of 3.94 picomolar and a lower affinity site (L) IC₅₀-L of 834 picomolar. [Wang et al., PLoS One. 3(2):e1554 (2008); Wang et al., PLoS One. 4(1):e4282 (2009).] The high affinity site was subsequently identified as the FLNA pentapeptide of SEQ ID NO: 1 (US Patent Publication 2009/0191579 and its predecessor application Ser. No. 60/985,086 that was filed on Nov. 2, 2007), whereas the lower affinity site has not yet been identified.

Morphinan and 4,5-epoxymorphinan ring compounds such as naloxone (NLX), naltrexone (NTX), nalorphine, nalbuphine and buprenorphine are commercially available products and bind well to the high affinity FLNA pentapeptide of SEQ ID NO: 1 (VAKGL). However, when used at a dosage recited on the product labels, those compounds also bind to the lower affinity site on FLNA, and typically also bind to the MOR. Some of the compounds are MOR antagonists such as naloxone, naltrexone, nalbuphine, whereas others such as buprenorphine and etorphine are full or partial agonists of MOR.

Binding to that lower affinity FLNA site impairs the activity of the FLNA protein to exhibit its activities as discussed, utilized and illustrated herein. As a consequence, a FLNA-binding benzazocine-ring compound such as naloxone, naltrexone, nalorphine, nalbuphine, buprenorphine and similar compounds that also bind to the lower affinity site on the FLNA protein are utilized in an amount that is about 1000^{th} to about 1,000,000^{th} less than that amount normally used or suggested for use, and more preferably at a level that is about 10,000^{th} to about 100,000^{th} the minimal dosage listed on the product labels, as noted previously.

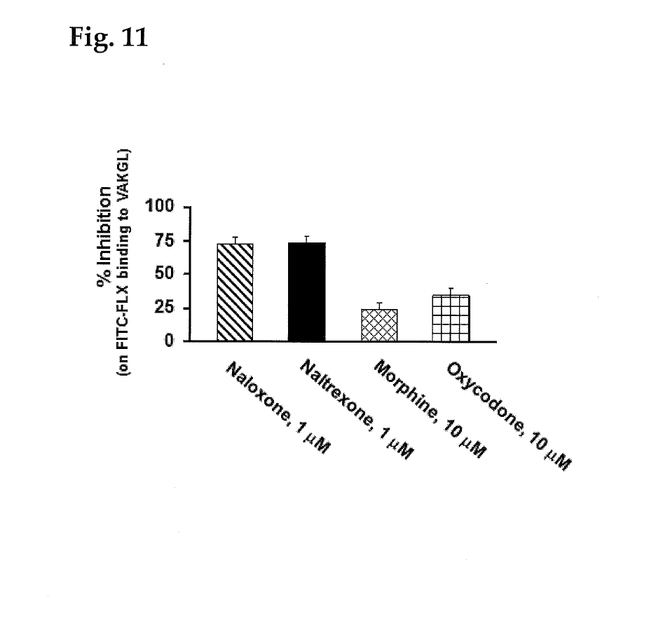

In addition, opioid agonists such as morphine itself and oxycodone do not bind well to the FLNA pentapeptide as is seen from the data of FIG. 11.

A FLNA-binding benzazocine-ring compound contemplated for use in the present invention inhibits the binding of fluorescein isothiocyanate-labeled naloxone (FITC-NLX) or tritiated-naloxone ([H³]NLX) to biotin-linked SEQ ID NO: 1 (Bn-VAKGL) bound to coated streptavidin plates under conditions described hereinafter in Example 1 to an extent that is at least about 60 percent and more preferably at least about 70 percent of the value obtained of the value obtained when present at a 10 μM concentration and using naloxone as the control inhibitor at the same concentration as the contemplated FLNA-binding benzazocine-ring compound, and up to about twice the value obtained with naloxone as control.

Naltrexone (NTX) can also be used as a control inhibitor. Average inhibition values obtained using NTX rather than NLX tend to be 1 or 2 percentage digits lower in absolute value than those with NLX. Thus, for example, where an average inhibition value at a particular concentration of NLX is 40 percent, one can expect values obtained with NTX to be about 38 or 39 percent. The binding inhibition values for a contemplated FLNA-binding benzazocine-ring compound are determined taking the expected NLX/NTX value difference into account.

Most of the FLNA-binding benzazocine-ring compounds are chiral and can exist as enantiomers of each other. It is presently preferred to utilize an enantiomeric compound that exhibits the lesser analgesic activity over the enantiomer that is more analgesically potent for the present method.

Another related class of useful FLNA-binding benzazocine-ring compounds that share the 2,6-methano-3-benzazocine ring structure shown are the benzomorphans that include zenazocine, volazocine, tonazocine, quadazocine, phenazocine, pentazocine, moxazocine, metazocine, ketazocine, ibazocine, gemazocine, fluorophen, eptazocine, dezocine, cogazocine, cyclazocine, brenazocine, anazocine and alazocine. These compounds are typically partial agonists of opioid receptors and are less preferred than are the morphinan or 4,5-epoxymorphinan ring compounds noted in the table above. The structures of some of these benzomorphans are illustrated below.

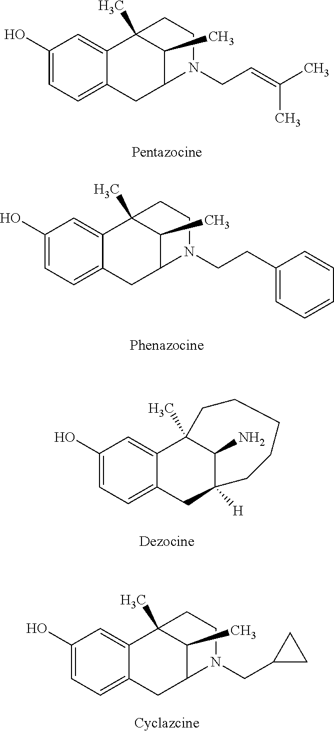 [SMILES: CC(C)=CCN1CC[C@@]2(C)c3cc(O)ccc3CC1[C@@H]2C ; C[C@H]1C2Cc3ccc(O)cc3[C@]1(C)CCN2CCc1ccccc1 ; C[C@@]12CCCCC[C@@H](Cc3ccc(O)cc31)[C@@H]2N ; C[C@H]1C2Cc3ccc(O)cc3[C@]1(C)CCN2CC1CC1]

### Pharmaceutical Compositions

A contemplated FLNA-binding benzazocine-ring compound useful in the invention can be provided for use by itself, or as a pharmaceutically acceptable salt. Regardless of whether in the form of a salt or not, a contemplated composition is preferably dissolved or dispersed in a pharmaceutically acceptable diluent that forms a pharmaceutical composition, and that pharmaceutical composition is administered the CNS and/or other cells.

A contemplated FLNA-binding benzazocine-ring compound can be used in the manufacture of a medicament (pharmaceutical composition) that is useful at least for inhibiting tau protein hyperphosphorylation in mammalian cells and mammalian cell preparations. A contemplated FLNA-binding benzazocine-ring compound can be used in the manufacture of a medicament that is useful at least for inhibiting the interaction of FLNA with α7nAChR and TLR4, as well as of Aβ₄₂ with α7nAChR in mammalian cells and mammalian cell preparations.

A contemplated pharmaceutical composition contains an effective amount of a contemplated FLNA-binding benzazocine-ring compound or a pharmaceutically acceptable salt thereof dissolved or dispersed in a physiologically tolerable carrier. Such a composition can be administered to mammalian cells in vitro as in a cell culture, or in vivo as in a living, host mammal in need.

A contemplated composition is typically administered a plurality of times over a period of days. More usually, a contemplated composition is administered once or twice daily. It is contemplated that once administration of a contemplated FLNA-binding benzazocine-ring compound has begun, the compound is administered chronically for the duration of the study being carried out, until tau hyperphosphorylation is no longer enhanced over a normal amount, the amount of hyperphosphorylation is reduced to a desired amount, and/or until the amount of one or more of the TLR4 activation protein markers is at background levels.

A contemplated compound can bind to FLNA at a 100 femtomolar concentration and effectively inhibit cytokine release from LPS-stimulated astrocytes in vitro. A contemplated compound is more usually utilized at picomolar to micromolar amounts. Thus, another way to achieve an effective amount of a contemplated FLNA-binding benzazocine-ring compound present in a contemplated pharmaceutical composition is to provide an amount that provides a FLNA-binding benzazocine-ring compound concentration of about 100 femtomolar to about micromolar to a host animal's blood stream or to an in vitro cell medium in practicing a contemplated method of the invention. A more usual amount is about picomolar to about micromolar. A still more usual amount is about picomolar to about nanomolar. A skilled worker can readily determine an appropriate dosage level of a contemplated FLNA-binding benzazocine-ring compound to inhibit a desired amount of tau protein hyperphosphorylation.

A contemplated pharmaceutical composition can be administered orally (perorally), parenterally, by inhalation spray in a formulation containing conventional nontoxic pharmaceutically acceptable carriers, adjuvants, and vehicles as desired. The term parenteral as used herein includes subcutaneous, intravenous, intramuscular, intrasternal injections, or infusion techniques. Formulation of drugs is discussed in, for example, Hoover, John E., Remington's Pharmaceutical Sciences, Mack Publishing Co., Easton, Pa.; 1975 and Liberman, H. A. and Lachman, L., Eds., Pharmaceutical Dosage Forms, Marcel Decker, New York, N.Y., 1980.

As already noted, a contemplated FLNA-binding benzazocine-ring compound is typically commercially available. Inasmuch as a contemplated pharmaceutical composition utilizes a far lesser amount of FLNA-binding benzazocine-ring compound than the commercially used amount of that active agent, a commercially available composition can be diluted appropriately for use herein. Thus, if the commercial product is a tablet, the commercial tablet can be crushed and then diluted with more of the same or similar diluent. Similarly, where the commercially available FLNA-binding benzazocine-ring compound is formulated to be a liquid for parenteral administration, that parenteral product can be diluted appropriately with normal saline, or other pharmaceutically acceptable liquid diluent. It is preferred that a contemplated pharmaceutical composition be prepared ab initio rather than by reformulating a commercially available product.

For injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution, phosphate-buffered saline. Liquid pharmaceutical compositions include, for example, solutions suitable for parenteral administration. Sterile water solutions of an active component or sterile solution of the active component in solvents comprising water, ethanol, or propylene glycol are examples of liquid compositions suitable for parenteral administration.

In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

Solid dosage forms for oral administration can include capsules, tablets, pills, powders, and granules. In such solid dosage forms, a contemplated compound is ordinarily combined with one or more excipients appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets, capsules and pills can additionally be prepared with enteric coatings.

A mammal in need of treatment and to which a pharmaceutical composition containing a contemplated FLNA-binding benzazocine-ring compound is administered can be a primate such as a human, an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like. Where in vitro mammalian cell contact is contemplated, a CNS tissue culture of cells from an illustrative mammal is often utilized, as is illustrated hereinafter.

Preferably, the pharmaceutical composition is in unit dosage form. In such form, the composition is divided into unit doses containing appropriate quantities of the FLNA-binding benzazocine-ring compound as active agent. The unit dosage form can be a packaged preparation, the package containing discrete quantities of the preparation, for example, in vials or ampules.

Several useful contemplated FLNA-binding benzazocine-ring compounds are amines and can typically be used in the form of a pharmaceutically acceptable acid addition salt derived from an inorganic or organic acid. Exemplary salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate and undecanoate.

The reader is directed to Berge, J. Pharm. Sci. 68(1):1-19 (1977) for lists of commonly used pharmaceutically acceptable acids and bases that form pharmaceutically acceptable salts with pharmaceutical compounds.

In some cases, the salts can also be used as an aid in the isolation, purification or resolution of the compounds of this invention. In such uses, the acid used and the salt prepared need not be pharmaceutically acceptable.

### Example 1

### FITC-NLX-Based FLNA Screening Assay

A series of binding studies was carried out using various potential a FLNA-binding compounds as ligand and the FLNA pentapeptide of SEQ ID NO: 1 as the receptor. The assay described below is one basis for defining a FLNA-binding benzazocine-ring compound contemplated for use in the present invention, and is the assay of Example 1 noted previously herein. The specifics of this assay are set out below.

A. Streptavidin-Coated 96-Well Plates

Streptavidin-coated 96-well plates (Reacti-Bind™ NeutrAvidin™ High binding capacity coated 96-well plate, Pierce-ENDOGEN) are washed three times with 200 μl of 50 mM Tris HCl, pH 7.4 according to the manufacturer's recommendation.

B. N-Biotinylated VAKGL Pentapeptide VAKGL) (SEQ ID NO: 1)

Bn-VAKGL peptide (0.5 mg/plate) is dissolved in 50 μl DMSO and then added to 4450 μl of 50 mM Tris HCl, pH 7.4, containing 100 mM NaCl and protease inhibitors (binding medium) as well as 500 μl superblock in PBS (Pierce-ENDOGEN) [final concentration for DMSO: 1%].

C. Coupling of Bn-VAKGL Peptides to Streptavidin-Coated Plate

The washed streptavidin-coated plates are contacted with 5 μg/well of Bn-VAKGL (100 μl) for 1 hour (incubated) with constant shaking at 25° C. [50 μl of Bn-VAKGL peptide solution from B+50 μl binding medium, final concentration for DMSO: 0.5%]. At the end of the incubation, the plate is washed three times with 200 μl of ice-cold 50 mM Tris HCl, pH 7.4.

D. Binding of FITC-Tagged Naloxone [FITC-NLX] to VAKGL.

Bn-VAKGL coated streptavidin plates are incubated with 10 nM fluorescein isothiocyanate-labeled naloxone (FITC-NLX; Invitrogen) in binding medium (50 mM Tris HCl, pH 7.4 containing 100 mM NaCl and protease inhibitors) for 30 minutes at 30° C. with constant shaking. The final assay volume is 100 μl. At the end of incubation, the plate is washed twice with 100 μl of ice-cold 50 mM Tris, pH 7.4. The signal, bound-FITC-NLX is detected using a DTX-880 multi-mode plate reader (Beckman).

E. Screening of Medicinal Chemistry Analogs

The potential FLNA-binding benzazocine-ring compounds to be assayed are first individually dissolved in 25% DMSO containing 50 mM Tris HCl, pH 7.4, to a final concentration of 1 mM (assisted by sonication when necessary) and then plated into 96-well compound plates. To screen new compounds, each compound solution (1 μl) is added to the Bn-VAKGL coated streptavidin plate with 50 μl/well of binding medium followed immediately with addition of 50 μl of FITC-NLX (total assay volume/well is 100 μl). The final screening concentration for each compound is 10 μM.

Each screening plate includes vehicle control (total binding) as well as naloxone (NLX) enantiomers [(−)NLX and (+)NLX] and/or naltrexone (NTX) as positive controls. Compounds are tested in triplicate, quadruplicate or quintuplicate. Percent inhibition of FITC-NLX binding for each compound is calculated [(Total FITC-NLX bound in vehicle−FITC-NLX bound in compound)/Total FITC-NLX bound in vehicle]×100%]. To assess the efficacies and potencies of the selected FLNA-binding benzazocine-ring compounds, compounds that achieve approximately 60-70% inhibition at 100 μM are screened further at 1 and 0.1 μM concentrations.

The results of this assay are shown in the Table below for NLX and NTX.

| | |
|---|---|
| FLNA-binding | Concentration of FLNA-binding Compound |

| Compound | 0.01 μM | 0.1 μM | 1 μM |
|---|---|---|---|
| (+)NLX | 40.9% | 41.7% | 46.8% |
| NTX | 40.20% | 47.40% | 49.10% |
| NTX | 37.9% | 46.5% | 55.4% |
| NTX | 37.9% | 46.5% | 55.4% |
| NTX | 34.8% | 45.2% | 51.6% |
| NTX | 38.5% | 43.3% | 48.0% |
| (−)NLX | 42.50% | 54.20% | 56.20% |
| (−)NLX | 42.96% | 50.30% | 53.93% |
| (−)NLX | 41.9% | 48.6% | 51.5% |
| (−)NLX | 34.3% | 38.5% | 45.8% |
| (−)NLX | 41.4% | 47.2% | 50.2% |
| (+)NLX | 43.40% | 48.10% | 49.80% |
| (+)NLX | 41.74% | 49.95% | 58.62% |

Binding inhibition studies were carried out using several morphinan and 4,5-epoxymorphinan ring compounds. The percentages of inhibition at 10 μM and 1 μM concentrations are shown in the Table below.

| | |
|---|---|
| | Percent |
| FLNA-binding | Inhibition |

| Benzazocine-ring | 10 μM | 1 μM |
|---|---|---|
| Compound | (n = 4) | (n = 5) |
| β-Funaltrexamine | 69.4 | 49.8 |
| Natrindole | 65.0 | 41.0 |
| Levorphanol | 69.4 | 46.8 |
| 6-β-Naltrexol | 64.8 | 50.5 |
| Nalorphine | 60.2 | 58.7 |
| Nalbuphine | 76.2 | 65.4 |
| Buprenorphine | 75.4 | 72.7 |
| Diprenorphine | 65.8 | 45.9 |
| Nalmefene | 66.9 | 44.5 |
| Naloxone | ND* | 71.6 |
| Naltrexone | ND* | 76.4 |

*ND = Not done.

As is seen from the above results, the morphinan and 4,5-epoxymorphinan ring compounds other than naloxone and naltrexone also exhibited FLNA binding inhibition. Buprenorphine and nalbuphine inhibited binding to the FLNA pentapeptide about as much as did naltrexone and naloxone. The somewhat lessened inhibitory activity of the other morphinan and 4,5-epoxymorphinan ring compounds can be useful because blood levels of the inhibitor can be hard to monitor when extremely potent compounds such as naloxone and naltrexone are used.

### Example 2

### High-Affinity FLNA-Binding Compounds Reduce Aβ₄₂-Induced α7nAChR-FLNA Association, ERK2 Activation and Tau Hyperphosphorylation in Synaptosomes

Six high-affinity FLNA-binding compounds [A0040, A0068, B0055, C0137, C0138 and (+)naloxone (+NLX) whose structural formulas are shown below] were assayed to determine whether they could disrupt the association of FLNA and α7nAChR in synaptosomes prepared from frontal cortices of adult rats. Synaptosomes were exposed to 100 nM Aβ₄₂ for 30 minutes, and with 0.1 or 1 μM compounds added either simultaneously or 10 minutes earlier. Controls were a vehicle (no Aβ₄₂) and an Aβ₄₂ alone condition.

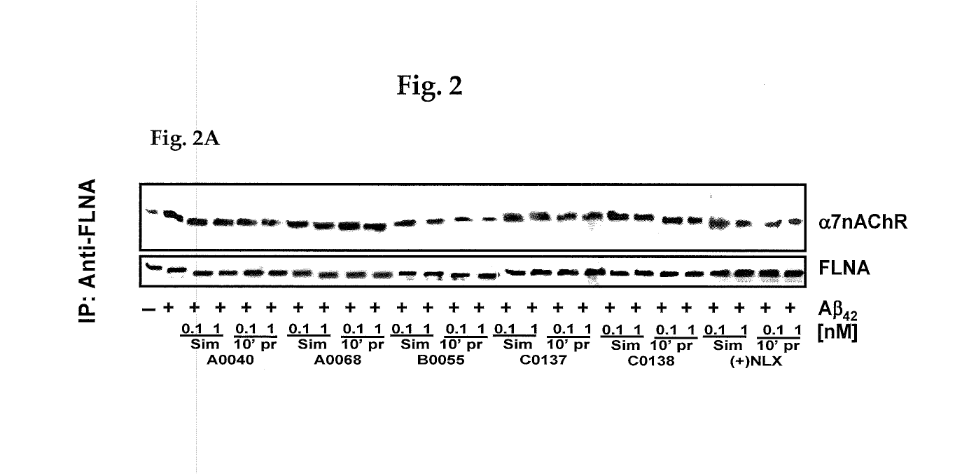

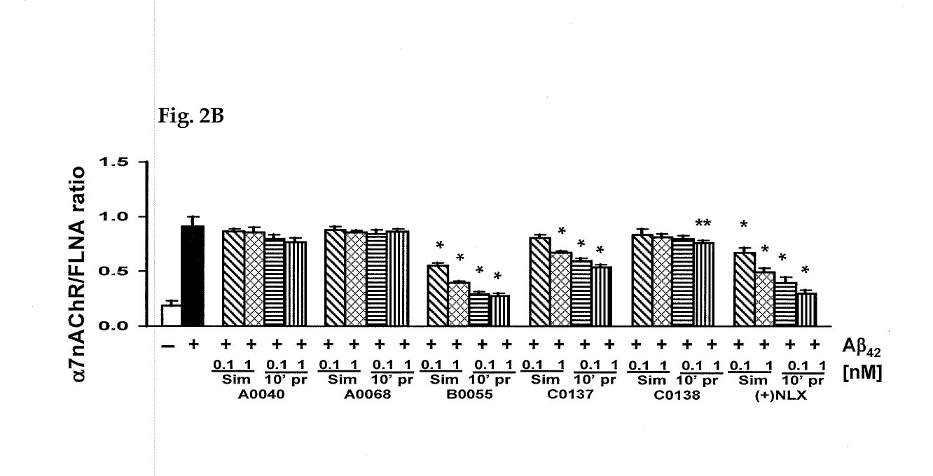

FIG. 2A shows the Western blots from all six compounds assayed, including (+)naloxone (NLX), as well as the quantitation of the blots (FIG. 2B). All six of those compounds reduced the α7nAChR-FLNA association with 10-minute pre-incubation, and Compound B0055 and +NLX also markedly reduced this coupling with simultaneous administration (FIG. 2B).

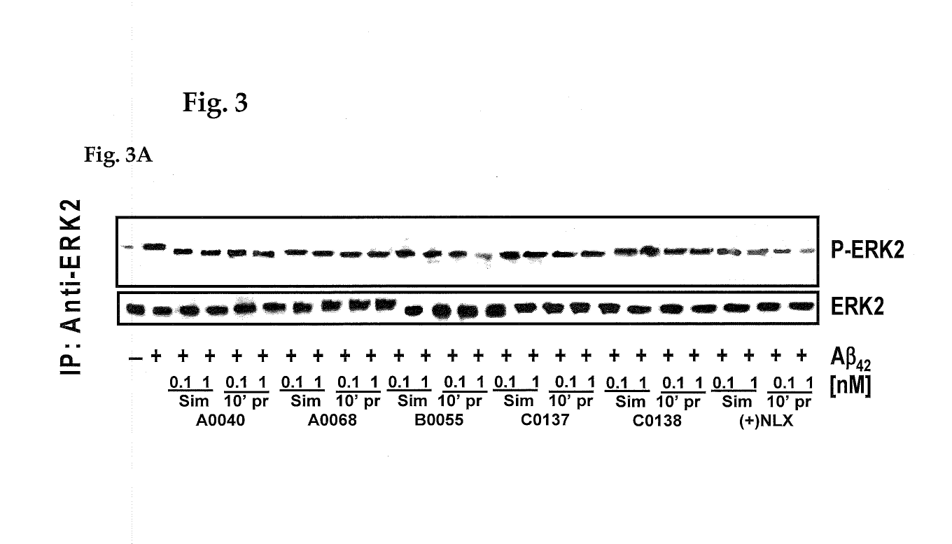

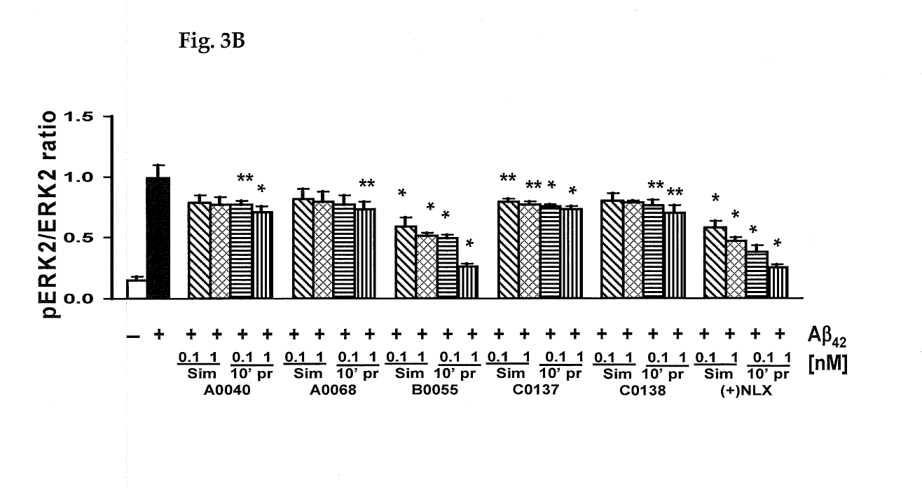

To assess Aβ₄₂ signaling via α7nAChR after compound administration, levels of phosphorylated ERK2 were measured in the same synaptosome preparations treated with Aβ₄₂ and the above compounds using Western blots (FIG. 3A). Phosphorylation of ERK2 indicates its activation, which leads to tau hyperphosphorylation. Compared to the control condition, Aβ₄₂ strongly activates ERK2. This activation is suppressed by all six compounds at 0.1 and 1 nM with 10 minute pretreatment, and also by Compound B0055 and (+)NLX with simultaneous treatment (FIG. 3B).

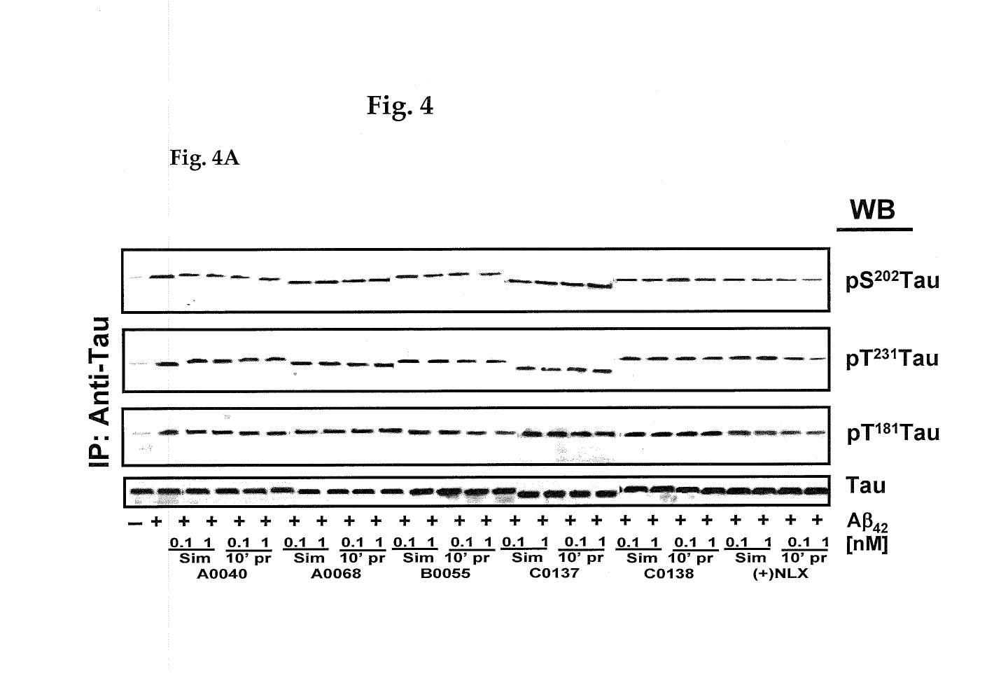

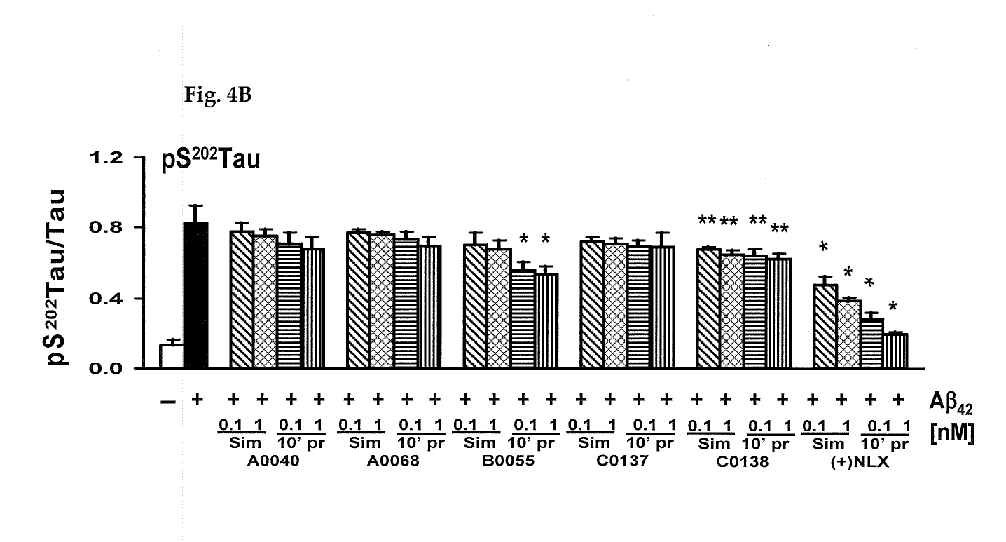

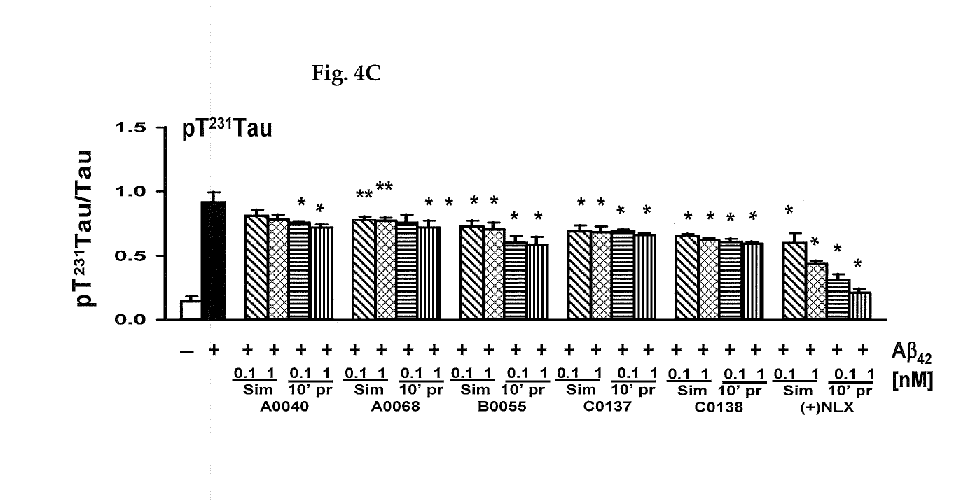

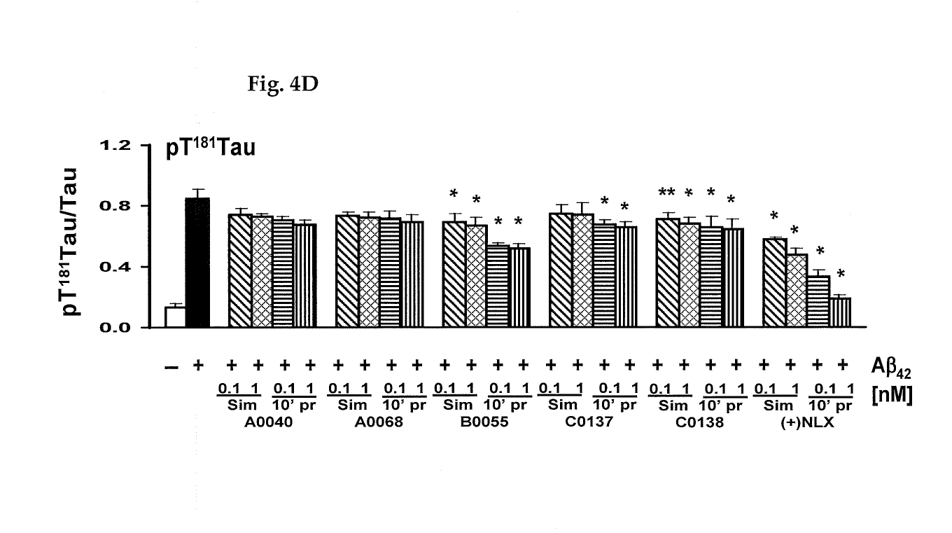

Next assessed was whether the FLNA-binding compounds also decrease hyperphosphorylation of tau, a downstream effect of Aβ₄₂ binding to α7nAChR and ERK2 activation. The three primary phosphorylation sites on tau protein were examined for their phosphorylation levels compared to total tau protein content using Western blots (FIG. 4A). Tau, phosphorylated at these three sites, is a constituent of NFTs. Consistent with effects on FLNA-α7nAChR association, and ERK2 activation, (+)NLX particularly decreased Aβ₄₂-induced hyperphosphorylation of tau at all three sites (FIG. 4D), with several of the other of the FLNA-binding compounds also decreasing Aβ₄₂-induced hyperphosphorylation, particularly following a 10 minute pre-incubation.

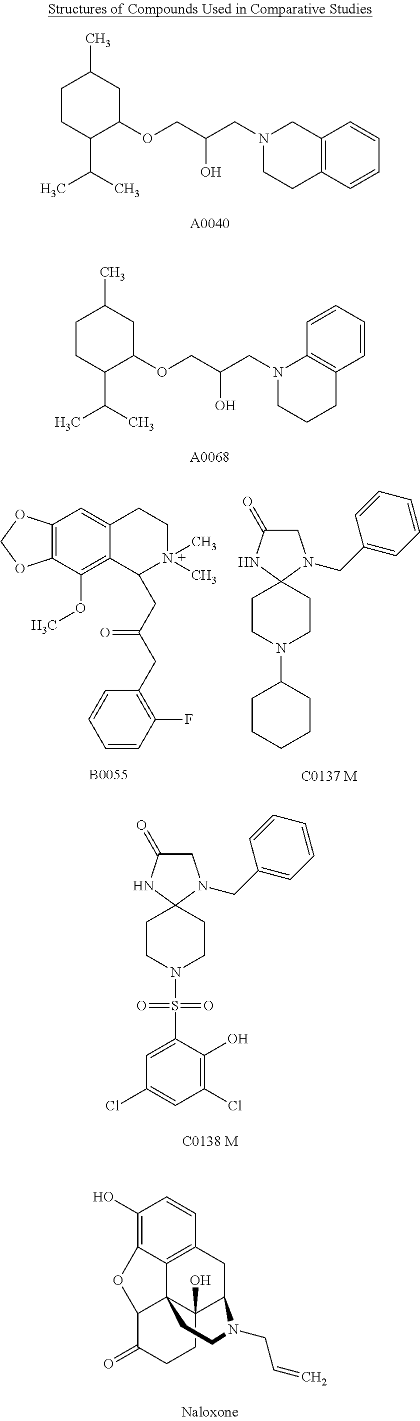

### Example 3

### FLNA Affinity Binding Study

A study was carried out as described in Wang et al., PLoS One. 3(2):e1554 (2008), FIG. 3, to determine the affinity of binding of [³H]NLX to the FLNA protein in the presence of NTX. The results of that binding study are illustrated in FIG. 1A herein.

More specifically, the competition (displacement) curve (FIG. 1) for the inhibition of [³H]NLX binding by naltrexone to membranes from FLNA-expressing A7 (human melanocytic; ATCC CRL-2500) cells that are free of most receptors and particularly mu shows two affinity sites with IC_{50-H} (high) of 3.94 picomolar and IC_{50-L} (low) of 834 picomolar, indicating an affinity difference of about 200-fold between the high- and low-affinity binding sites. A nonlinear curve-fit analysis was performed using a competition equation that assumed two saturable sites for the naltrexone curve comprising of 16 concentrations ranging from 0.1 pM to 1 mM. Data are derived from six studies each using a different set of A7 cells.

A similar study was carried out using FITC-labeled NLX (FITC-NLX) in place of [³H]NLX, as is shown in FIG. 1A. The results of that study are similar in that two affinity sites are noted with IC_{50-H} (high) of 1.63 picomolar and IC_{50-L} (low) of 18,800 picomolar, indicating an affinity difference of almost 12.000-fold between the high- and low-affinity binding sites.

### Example 4

### Studies With Morphine and Oxycodone

Studies were carried out comparing the effects of the MOR agonists morphine and oxycodone in a binding inhibition study using FITC-labeled naloxone bound to the biotinylated pentapeptide of FLNA of SEQ ID NO: 1. Thus, 10 nM FITC-conjugated NLX was bound to biotinylated-VAKGL peptides of SEQ ID NO: 1 at 0.5 mg/well coated onto streptavidin-coated 96-well plates (n=5). Following the reaction, the incubation medium was removed, plates were washed and the percent inhibitions of the assayed compounds were calculated by comparing to vehicle control. The results are shown in FIG. 11 in which it seen that whereas 1 μM NLX and 1 μM NTX each inhibited the binding of FITC-labeled NLX by about 75 percent, 10 μM (ten-fold greater) concentrations of either morphine or oxycodone were required to provide about 25 to about 50 percent inhibition.

Each of the patents, patent applications and articles cited herein is incorporated by reference. The foregoing description and the examples are intended as illustrative and are not to be taken as limiting. Still other variations within the spirit and scope of this invention are possible and will readily present themselves to those skilled in the art.

1. A method of inhibiting hyperphosphorylation of the tau protein that comprises the steps of administering to cells of the central nervous system in recognized need a FLNA-binding effective amount of a FLNA-binding benzazocine-ring compound or a pharmaceutically acceptable salt thereof that binds to a pentapeptide of filamin A (FLNA) of SEQ ID NO: 1, and inhibits at least about 60 percent of the FITC-labeled naloxone binding when present at a 10 μM concentration and using unlabeled naloxone as the control inhibitor at the same concentration.

2. The method according to claim 1, wherein said FLNA-binding benzazocine-ring compound is a morphinan or 4,5-epoxymorphinan ring compound.

3. The method according to claim 2, wherein said morphinan or 4,5-epoxymorphinan ring compound is naloxone or naltrexone.

4. The method according to claim 2, wherein said morphinan or 4,5-epoxymorphinan ring compound is other than naloxone or naltrexone.

5. The method according to claim 1, wherein said FLNA-binding benzazocine-ring compound or a pharmaceutically acceptable salt thereof is present dissolved or dispersed in a pharmaceutically acceptable diluent as a pharmaceutical composition when administered.

6. The method according to claim 1, wherein said administration is carried out a plurality of times.

7. The method according to claim 6, wherein said administration is carried out daily.

8. The method according to claim 6, wherein said administration is carried out multiple times daily.

9. The method according to claim 5, wherein said pharmaceutical composition is in liquid form.

10. The method according to claim 5, wherein said pharmaceutical composition is in solid form.

11. The method according to claim 1, wherein said FLNA-binding benzazocine-ring compound contains at least four of the six pharmacophores of FIGS. 5-10.

12. The method according to claim 1, wherein said FLNA-binding benzazocine-ring compound is a) an opioid receptor antagonist compound, b) a mixed opioid receptor agonist and antagonist (agonist/antagonist) compound, c) an opioid receptor agonist compound or d) an enantiomer of an opioid receptor antagonist, agonist/antagonist or agonist compound.

13. A method of inhibiting a TLR4-mediated immune response that comprises administering to TLR4-containing cells in recognized need thereof a FLNA-binding effective amount of a FLNA-binding benzazocine-ring compound benzazocine-ring compound or pharmaceutically acceptable salt thereof that binds to a pentapeptide of filamin A (FLNA) of SEQ ID NO: 1, and inhibits at least about 60 percent of the FITC-labeled naloxone binding when present at a 10 μM concentration and using unlabeled naloxone as the control inhibitor at the same concentration.

14. The method according to claim 13, wherein said FLNA-binding benzazocine-ring compound is a morphinan or 4,5-epoxymorphinan ring compound.

15. The method according to claim 14, wherein said morphinan or 4,5-epoxymorphinan ring compound is naloxone or naltrexone.

16. The method according to claim 14, wherein said morphinan or 4,5-epoxymorphinan ring compound is other than naloxone or naltrexone.

17. The method according to claim 13, wherein said FLNA-binding benzazocine-ring compound or a pharmaceutically acceptable salt thereof is present dissolved or dispersed in a pharmaceutically acceptable diluent as a pharmaceutical composition when administered.

18. The method according to claim 13, wherein said administration is carried out a plurality of times.

19. The method according to claim 18, wherein said administration is carried out daily.

20. The method according to claim 18, wherein said administration is carried out multiple times daily.

21. The method according to claim 17, wherein said pharmaceutical composition is in liquid form.

22. The method according to claim 17, wherein said pharmaceutical composition is in solid form.

23. The method according to claim 13, wherein said FLNA-binding benzazocine-ring compound contains at least four of the six pharmacophores of FIGS. 5-10.

24. The method according to claim 13, wherein said FLNA-binding benzazocine-ring compound is a) an opioid receptor antagonist compound, b) a mixed opioid receptor agonist and antagonist (agonist/antagonist) compound, c) an opioid receptor agonist compound or d) an enantiomer of an opioid receptor antagonist, agonist/antagonist or agonist compound.
